(19) United States
(12) Patent Application Publication
(10) Pub. No.: US 2013/0339043 A1
(43) Pub. Date: Dec. 19, 2013

(54) VISION CORRECTION PRESCRIPTION AND HEALTH ASSESSMENT FACILITY

(71) Applicant: Advanced Vision Solutions, Inc., Carson City, NV (US)

(72) Inventors: Sherwyne R. Bakar, Palo Alto, CA (US); Mark Klusza, Houston, TX (US); James M. Janky, Los Altos, CA (US)

(21) Appl. No.: 13/917,301

(22) Filed: Jun. 13, 2013

Related U.S. Application Data
(60) Provisional application No. 61/659,183, filed on Jun. 13, 2012.

(51) Int. Cl. A61B 3/18; G06Q 10/10; G06Q 30/06; A61B 3/02; G06Q 50/22

(52) U.S. Cl. CPC A61B 3/185; A61B 3/02; G06Q 50/22; G06Q 30/0631; G06Q 10/1093
USPC 705/2; 351/223

(57) ABSTRACT

A self-contained, free-standing vision-based health assessment/vision correction prescription system is provided, with a variety of health assessment sensors and vision correction sensors offered in a single, self-contained, Internet-connected facility that allows automated fulfillment of items that address the needs of a user that are determined by a vision/health assessment.

### CROSS-REFERENCE TO RELATED APPLICATIONS

This application claims the benefit of copending U.S. Provisional Application No. 61/659,183, filed Jun. 13, 2012 “VISION CORRECTION PRESCRIPTION AND HEALTH ASSESSMENT FACILITY” which is hereby incorporated b reference in its entirety.

### BACKGROUND

This application relates to a system and method for determining various vision and ocular parameters for the purposes of providing a vision corrective prescription and/or providing a health condition assessment for the presence of health conditions determinable from such parameters, and more specifically to a low-cost screening unit for determining same.

### SUMMARY

In an embodiment, a self-contained, free standing vision-based health assessment/vision correction prescription system is described. It may be described as a health service facility, with a variety of health assessment sensors and vision correction sensor available for a user. In an embodiment, a suite of health assessment sensors is offered in a single facility. In an embodiment a suite of vision correction prescription sensors are offered, with means for fulfilling a variety of vision correction systems, managed via an online service wherein the facility has access to the Internet. In an embodiment, any combination of health assessment sensors and vision correction sensors can be combined in a single facility. The system may be mounted in a free standing package, such as a kiosk, and in embodiments may be accessible by either sitting or standing users. The system may be located in a professional services office, or in a publically accessible location such as a mall or a government service facility like a post office. The suite of sensors may comprise a number of individual sensors tailored for a single specific health care assessment function or a single vision correction prescription function, or several functions may be combined into a single sensor system.

In embodiments, the systems and methods disclosed herein may include a system comprising a self a self-contained, standing housing, configured to include an interface for a user, at least one vision assessment facility integrated with the housing, the vision assessment facility capable of determining a prescription for at least one of contact lenses and eyeglasses for the user, and at least one health assessment facility related to a non-vision aspect of the health of a user integrated with the housing. The system may include a vision assessment facility that automatically aligns with the eyes of the user, without requiring a mechanical element for positioning the head of the user relative to the vision assessment facility. The system may further include a vision recommendation for analyzing a need identified by the vision assessment facility and recommending at least one of an item and an action to address the need. The system may further include an electronic commerce module for allowing a user to order a recommended item. The recommended items may include contact lenses or eyeglasses. The system may further include a scheduling module for allowing a user to schedule an appointment with an eye specialist. The system may include an eye glass fitting module. The system may include a housing that is configured as a kiosk adapted to be located in a retail location. The system may include a network communication facility. The system may include a health recommendation module for analyzing a need identified by the health assessment facility and recommending at least one of an item and an action to address the need. The system may include an electronic commerce module for allowing a user to order a recommended item. The system may further include a scheduling module for allowing a user to schedule an appointment to address a recommended item. The system may include a plurality of health assessment facilities, wherein at least two of the facilities are disposed on a rotating carousel to allow serial presentation of the facilities to the user. The system may include a plurality of health assessment facilities, wherein at least two of the facilities are disposed to allow presentation of the facilities to the user without requiring a rotating carousel. The system may include a vision assessment facility that is capable of providing both contact lens and eyeglass prescriptions for the user. The system may include a health assessment facility which is selected from the group consisting of a contact lens auto refractor, a corneal mapper, a corneal spline generator, a retinal macular condition sensor, a retinal circulatory physiology sensor, a 3D surface scanner, a glaucoma sensor, a blood pressure monitor, a pulse rate monitor, a diabetes sensor, and an iris scan sensor.

The system may further include a payment system by which a user may pay for at least one of an assessment, a recommended item, and a recommended action. In embodiments, the systems and methods disclosed herein may include a network-connected, retail kiosk, comprising a plurality of health assessment facilities, each adapted to assess a health condition of a user, a vision assessment facility capable of determining a contact lens prescription and an eyeglass prescription of a user, the vision assessment facility adapted to align with the eyes of the user and assess vision while the head of the user remains in a natural, unconstrained position, a recommendation module for recommending at least one of an item and an action based on at least one of a health assessment and a vision assessment, an electronic commerce module for ordering a recommended item; and a scheduling module for scheduling a recommended action. The system may include a health assessment facility selected from the group consisting of a contact lens auto refractor, a corneal mapper, a corneal spline generator, a retinal macular condition sensor, a retinal circulatory physiology sensor, a 3D surface scanner, a glaucoma sensor, a blood pressure monitor, a pulse rate monitor, a diabetes sensor, and an iris scan sensor.

In embodiments, the systems and methods disclosed herein may comprise a computer readable medium containing program instructions wherein execution of the program instructions by one or more processors of a computer system causes the one or more processors to carry out certain steps. The steps may include conducting a vision assessment of a user via at least one vision assessment facility capable of determining a prescription for at least one of contact lenses and eyeglasses for the user, conducting a health assessment of a user via at least one health assessment facility related to a non-vision aspect of the health of a user, storing data obtained from the vision assessment and health assessment on a memory device, retrieving, in response to a user request via a user interface, requested vision assessment and health assessment data, and presenting the retrieved vision assessment and health assessment data to the user via the user interface. The computer readable medium may further comprise automatically aligning the vision assessment facility with the eyes of a user, without requiring a mechanical element for positioning the head of the user relative to the vision assessment facility. The systems and methods disclosed herein may further comprise conducting a vision recommendation for a need identified by the vision assessment and recommending at least one of an item and an action to address the need. The computer readable medium may include processing a payment by a user to order a recommended item. The computer readable medium may comprise scheduling an appointment with an eye specialist. The computer readable medium may comprise conducting a health recommendation for a need identified by the health assessment and recommending at least one of an item and an action to address the need. The computer readable medium may comprise processing a payment by a user to order a recommended item. The computer readable medium may comprise scheduling an appointment to address a recommended item.

### BRIEF DESCRIPTION OF THE FIGURES

The invention and the following detailed description of certain embodiments thereof may be understood by reference to the following figures:

FIG. 1A depicts a vision correction prescription system block diagram in an embodiment of the present invention using a carousel sensor suite system.

FIG. 1B depicts a vision correction prescription system block diagram in an embodiment of the present invention using a direct access sensor suite system.

FIG. 2A depicts a health condition assessment block diagram for a carousel sensor suite in an embodiment of the present invention.

FIG. 2B depicts a mounting system block diagram in an embodiment of the present invention using a direct access sensor suite system.

FIG. 2C depicts a combined health assessment/prescription system using a direct access sensor suite system.

FIG. 3A depicts a use example diagram with a seated user and a carousel sensor suite.

FIG. 3B depicts a use example diagram with a seated user and a direct access sensor suite.

FIG. 3C depicts a use example diagram with a standing user and a direct access sensor suite.

FIG. 3D depicts the User Interface Display with Direct Access Sensor Suite.

FIG. 3E depicts a Multiple Sensor Suite using a Single Lens.

FIG. 4A depicts a mounting and transport system configuration embodiment.

FIG. 4B depicts another mounting and transport system configuration embodiment.

FIG. 5 depicts a mounting and transport system alignment configuration embodiment.

FIG. 6A depicts a control system block diagram for the carousel sensor suite in an embodiment of the present invention.

FIG. 6B depicts a controls system block diagram for the direct access sensor suite in an embodiment of the invention.

FIG. 7 depicts a user identification flow chart in an embodiment of the present invention.

FIG. 8 depicts a user selection of service flow chart in an embodiment of the present invention.

FIG. 9 depicts a user payment for service flow chart in an embodiment of the present invention.

FIG. 10 depicts a user examination by selected sensor system and mounting and transport operation in an embodiment of the present invention.

FIG. 11A depicts a prescription generation flow chart in an embodiment of the present invention.

FIG. 11B depicts a prescription generation eyeglasses flow chart in an embodiment of the present invention.

FIG. 11C depicts a prescription generation eyeglasses frame flow charting an embodiment of the present invention.

FIG. 12 depicts a report preparation and delivery flow chart in an embodiment of the present invention.

While the invention has been described in connection with certain preferred embodiments, other embodiments would be understood by one of ordinary skill the art and are encompassed herein.

All documents referenced herein are hereby incorporated by reference.

### DETAILED DESCRIPTION

The present invention will now be described in detail by describing various illustrative, non-limiting embodiments thereof with reference to the accompanying drawings and exhibit. The invention may, however, be embodied in many different forms and should not be construed as being limited to the illustrative embodiments set forth herein. Rather, the embodiments are provided so that this disclosure will be thorough and will fully convey the concept of the invention to those skilled in the art. The claims should be consulted to ascertain the true scope of the invention.

In embodiments, the systems and methods disclosed herein may provide for a vision correction prescription or health condition assessment through analysis of an individual's eyes, and the like. In embodiments, the systems and methods disclosed herein may provide for a vision correction prescription system and a health condition assessment system. In embodiments the vision correction prescription system and health condition assessment system may comprise different analysis systems.

In embodiments, the vision correction prescription system may comprise, but is not limited to, an automatic prescription generator for contact lenses, which may further comprise, but is not limited to, an auto-refractor for determining a prescription for a contact lens set, a corneal mapping system for creating a good fit of contact lens to an individual's eyes, an automatic prescription generator for eyeglasses (such as based on an auto-refractor for determining the prescription, but not limited to this method), an automatic eyeglass frame/size analyzer for determining a size and shape of frames and lenses for the individual user, and the like.

In embodiments, the health condition assessment system may comprise a health condition assessment analysis system. In embodiments, the health condition assessment analysis system may, analyze an individual's eyes, (e.g. searching for signs of Glaucoma through measurement aqueous fluid pressure within the eye), measure blood sugar level for diabetes screening, analyze the macular condition of retina, analyze retinal circulatory physiology as an indicator of hypertension, conduct a user's eye lens condition assessment for indications of Alzheimer's disease, and the like.

Various embodiments are shown in the accompanying figures, which may include pictorial views and block diagrams. Embodiments of the systems and methods disclosed herein may make use of electronic subsystems in various testing devices, and incorporate sensors into individual modules that may be configured in the complete system for an automated presentation to the user.

In embodiments, the analysis systems deployed for each kind of assessment may be housed in a stand-alone housing designed for automated operation and activation by a user. In embodiments, the configuration of the housing may be that of a kiosk or the like, suitable for installation in a public place. In other embodiments, the configuration may be altered to suit the intended location, such as, but not limited to, in an office or service provider's facility. The service facility may be configured to accommodate a user in various physical positions, such as, but not limited to, standing or sitting. The system and housing may be referred to as a customer service kiosk, or as a health service facility, or a health service kiosk.

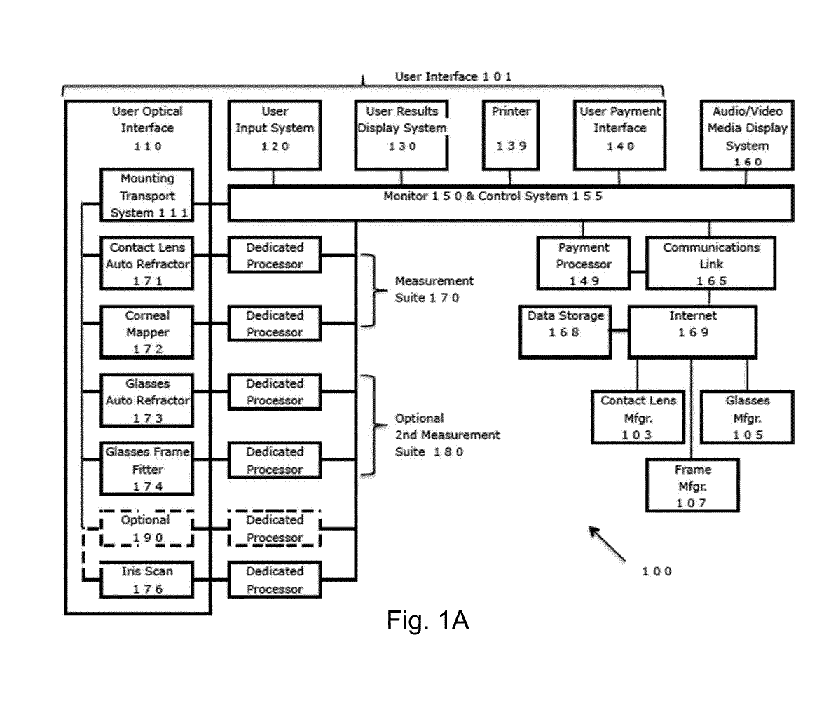

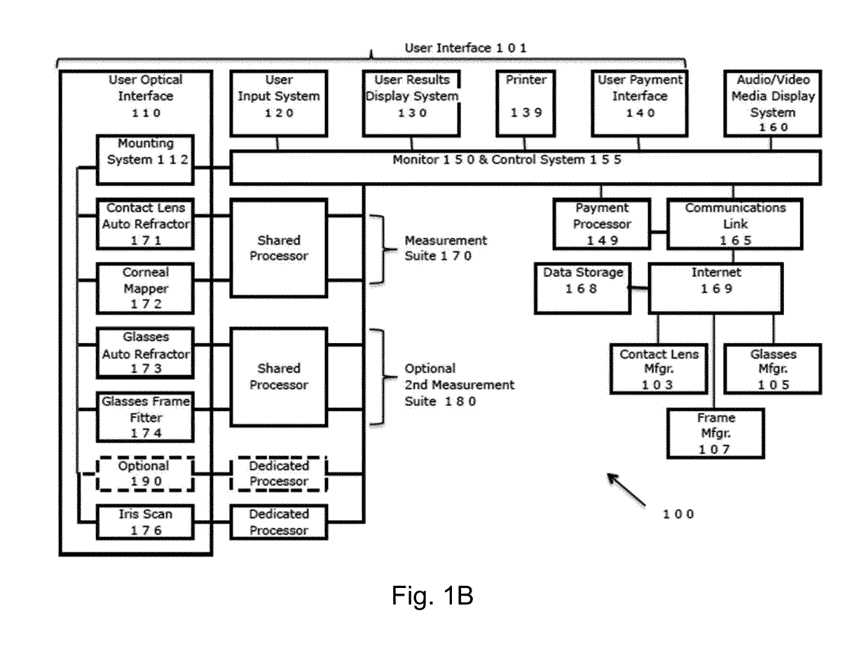

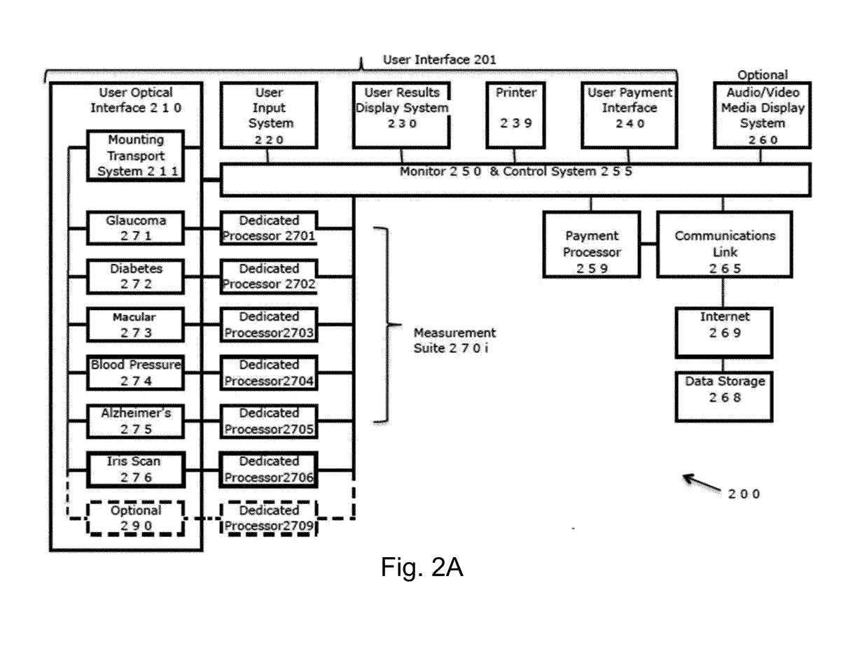

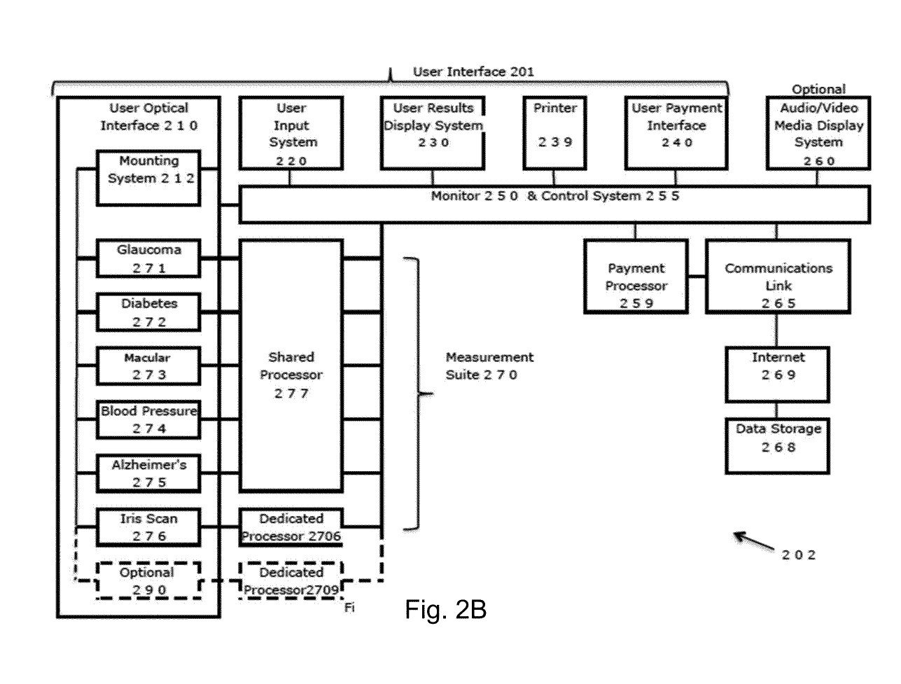

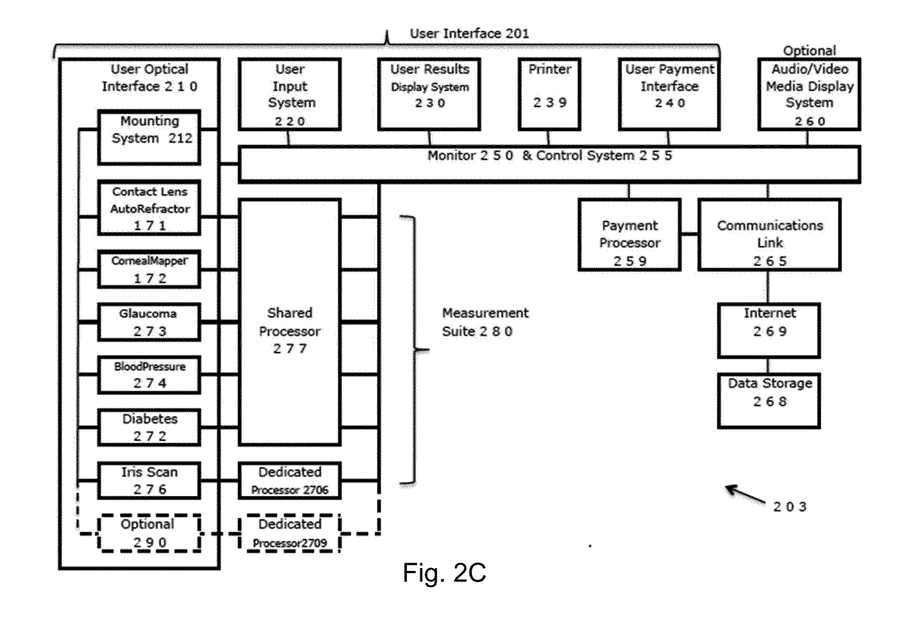

In embodiments, the systems and methods disclosed herein may comprise a vision correction prescription sensor suite. In a non-limiting example, the vision correction prescription sensor suite as shown in FIGS. 1A and 1B may comprise a contact lens auto refractor 171, a corneal mapper 172, a glasses auto refractor 173, a glasses frame fitter 174, an iris scan 176, and the associated dedicated processors for each application shown at 170 and 180. Similarly, in embodiments, the systems and methods disclosed herein may comprise a health assessment sensor suite. In a non-limiting example, as shown in FIGS. 2A and 2B, the health assessment sensor suit may comprise a glaucoma sensor 271, a diabetes sensor 272, a macular condition sensor 273, a blood pressure sensor 274, an Alzheimer's sensor 275, and an iris scan 276.

In embodiments, any of the health assessment sensors and any of the vision assessment prescription sensors may be integrated into a single health care facility, as shown in FIG. 2C at 203. The contact lens auto refractor 171, the corneal mapper 172, the glaucoma sensor 273, the blood pressure/pulse rate monitor 274, the diabetes sensor 272, and the iris scan sensor 276 may be installed in a single service facility/kiosk. Other embodiments, such as, but not limited to, the embodiments provided by FIG. 1A, FIG. 1B, FIG. 2A, and FIG. 2B, may use the same or similar control systems, processors and software to perform the analysis and prescription definition.

In embodiments, the systems and methods disclosed herein may comprise a housing for the various sensor components and electronic systems, a user interface with an analysis module and a display screen, a user input appliance, and the like. The system may be designed to provide a high level of flexibility in the configuration of the health assessment subsystems. A customer for the health assessment system may be able to arrange for any combination of assessment subsystems.

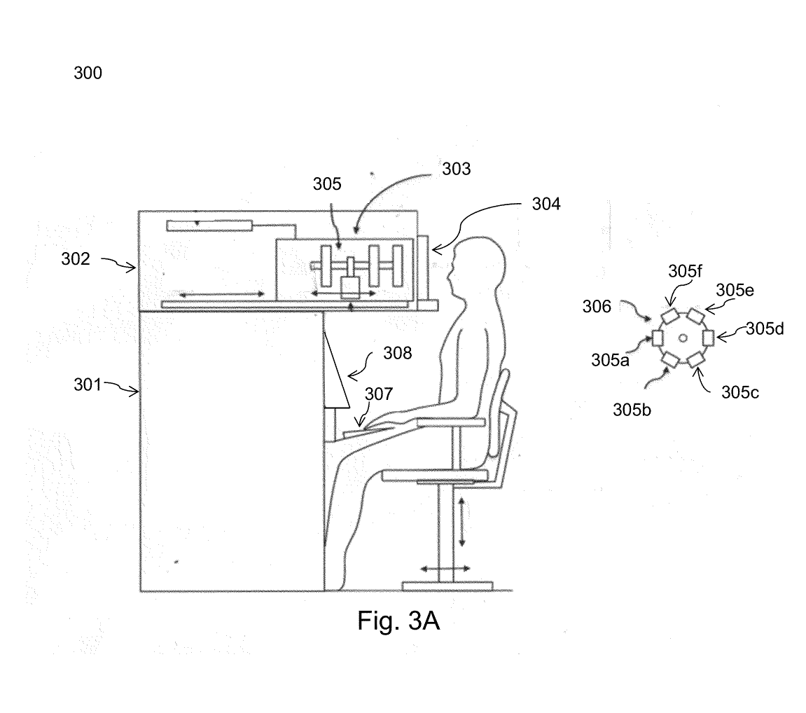

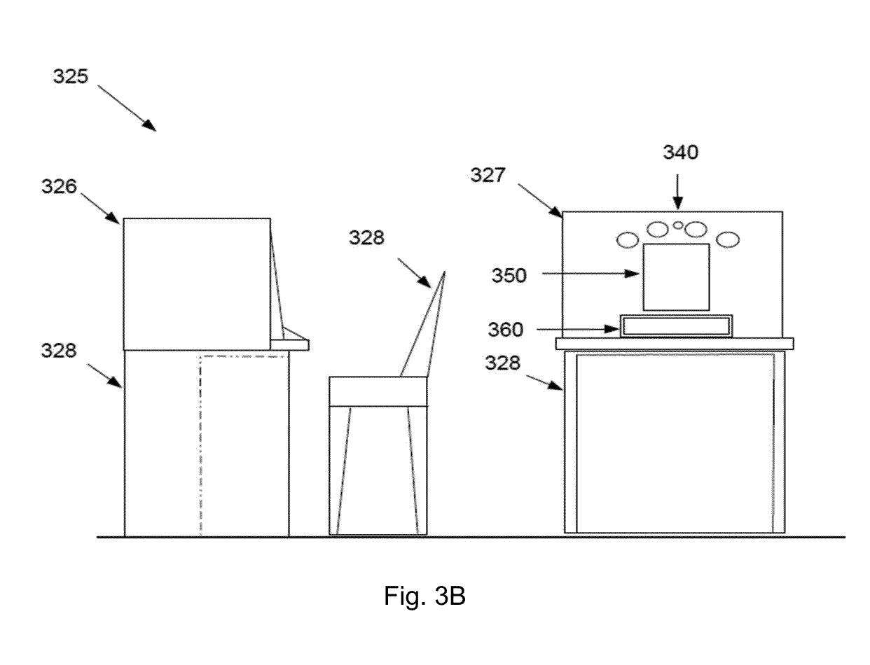

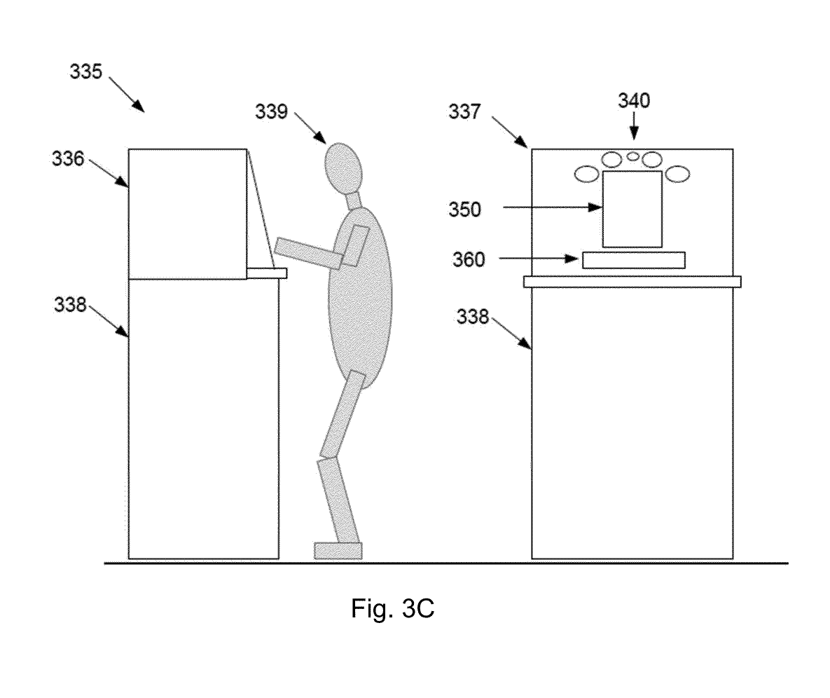

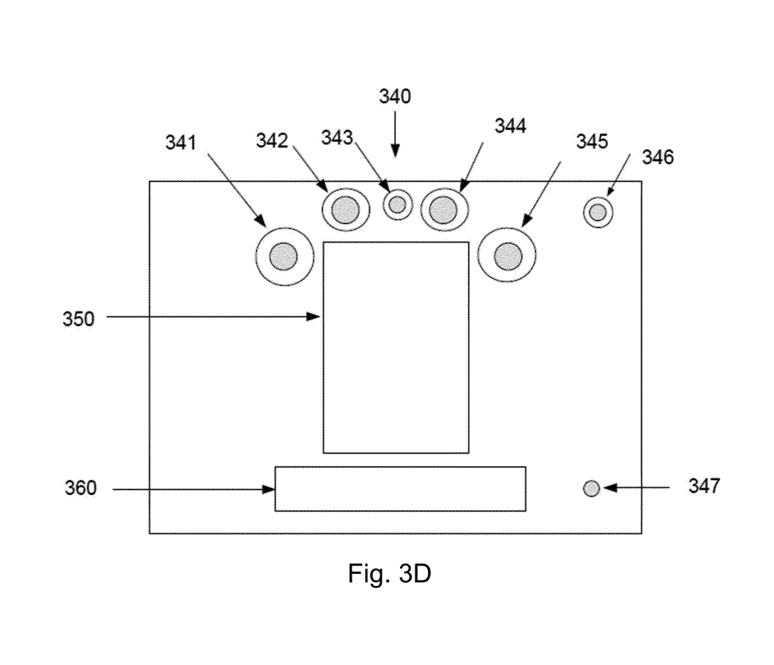

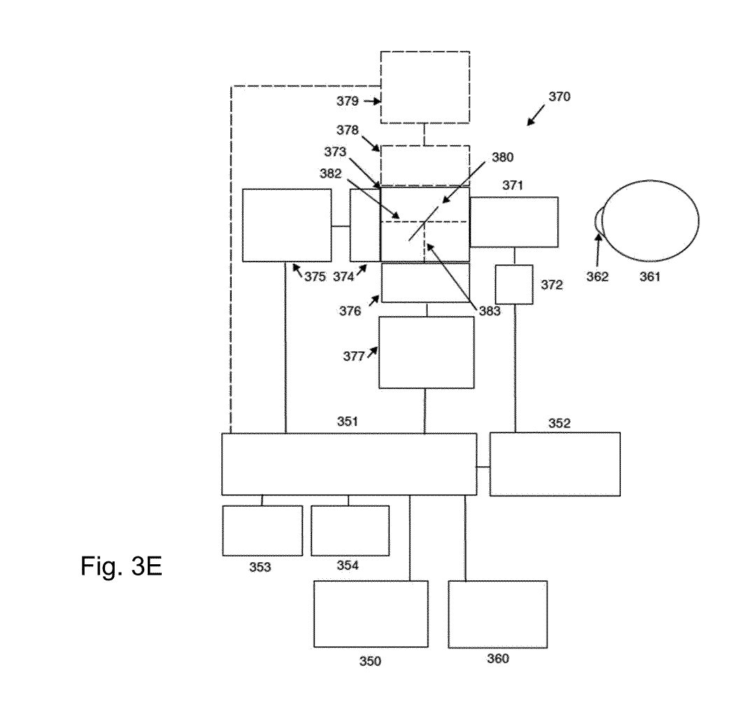

In embodiments and in FIG. 3A the systems and methods disclosed herein may comprise a health service facility 300 that may interact with a seated user. The health service facility 300 may comprise a base 301 that supports a carousel sensor suite system 302 configured to deliver selected sensors to the user's eyes at a user interface aperture, which may also be called the examination window 304. The base 301 may comprise the support electronics and dedicated processors, an example of which may be seen in FIGS. 1A and 1B. The carousel 303 may comprise a plurality of suitable sensors 305 in side view, and at 306 in a top view of a rotating wheel system. In embodiments, the rotating wheel system may bring the selected sensor towards the user interface aperture, according to a selection system based on the user input system 120 and user-activated control system 155. The sensors mounted on the rotating wheel 306 may comprise an iris scanner 305a, an auto-refractor 305b, a corneal mapper 305c, a glucose sensor 305d, a retina analyzer 305e, or an eyeglasses fitting sensor 305f, and the like as shown in FIGS. 1A, 1B, 2A and 2B, and as described in other portions of this application. In embodiments and in FIG. 2A, the health assessment system sensor modules may comprise a glaucoma sensor 271.

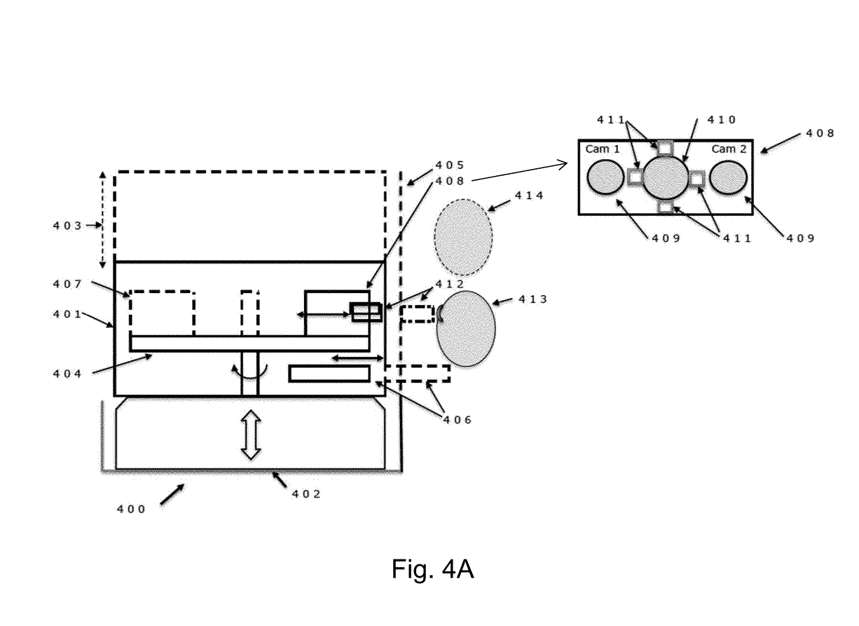

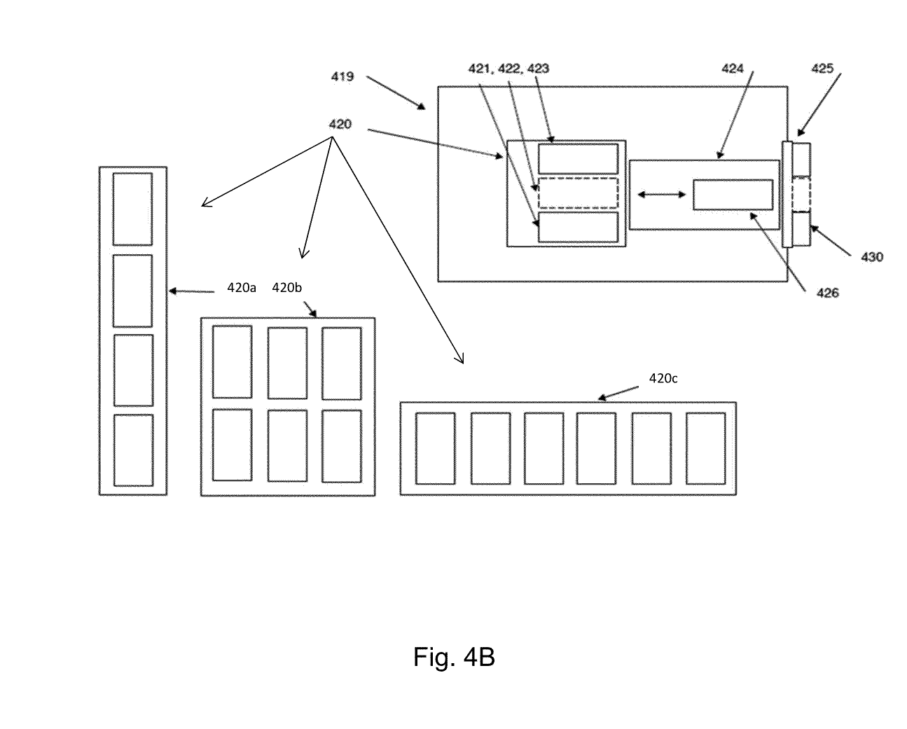

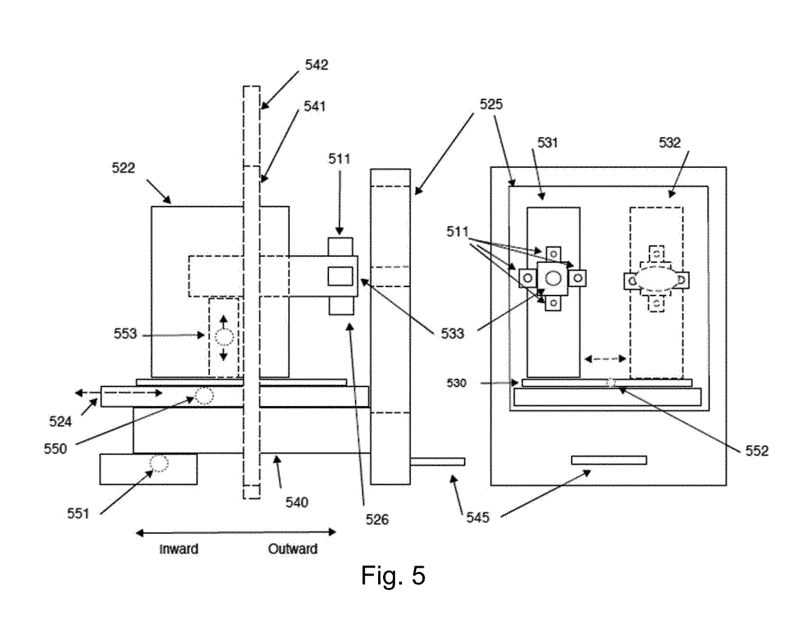

In embodiments and in FIG. 3A, the user may interact with the system via a keyboard 307 and view a display 308 located below the examination window 304. Embodiments Details of the sensor mounting and transport system are shown in FIGS. 4A, 4B, and 5. In embodiments and in FIG. 3B, the sensor suite may be mounted directly to the user interface 327 of the housing 326. In this embodiment, as shown at 325 in FIG. 3B for a sitting user and at 335 in FIG. 3C for a standing user, the suite of sensor(s) 340 may be mounted in juxtaposition to a display screen 350 which may also have a touchscreen for user input, as shown in FIGS. 3B, 3C, and 3D. In embodiments, a computer keyboard 360 may be used for accepting user inputs to the system.

In embodiments, the sensor suite shown at 340 in FIGS. 3B, 3C, and 3D may contain sensors configured for a single specific purpose, such as, but not limited to, auto-refraction or glucose monitoring, or may contain a sensor with multiple capability, depending on the analysis software associated with the problem being addressed. The sensor module may comprise a lens system for focusing on various parts of the eye, an image capture system comprising a Charge Coupled Device (“CCD”) commonly found in digital cameras, and various filters to isolate different wavelengths of light that are germane to the particular analysis of the sensor module. The lens system may be focused on any part of the eye, including the cornea, the aqueous humor liquid between the cornea and the iris, the lens of the eye, the vitreous humor in the interior of the eyeball, and the retina or the macular region of the retina. In embodiments, the lens system of a sensor may be configured for a specific eye part. In embodiments, the lens system of a sensor may be adjustable in order to access any one or more eye part in combination with each other. In embodiments, the dedicated processor may be configured to operate with more than one sensor data collection system and more than one sensor data analysis system. In a non-limiting example, the iris scan and the corneal mapper may use the same sensor to obtain data about the iris and the external surface of the cornea, respectively. In the same manner, the macular condition measurement system and the blood pressure measurement system may be realized with the same optical sensor, but the data obtained may be processed by different applications, while utilizing a shared processor. In embodiments and in FIG. 2A, the blood pressure sensor 274 may examine other parts of the user's face, to capture dynamic variations in skin deformation due to pulsating blood passing through veins and capillaries.

In embodiments and in FIG. 3D, the systems and methods described herein may comprise a user interface with a Direct Access Sensor Suite 340. The sensors in the sensor suite may comprise any or all of the sensors recited in FIG. 1B for vision correction prescription system, or any or all of the sensors recited in FIG. 2B for the health assessment system, or the like. The sensor suite for vision correction may comprise any or all of a contact lens auto-refractor 271, which is shown at 341 in FIG. 3D, a corneal mapper 272, shown at 342, a glasses auto refractor 273 shown at 343, a glasses frame fitter 374, shown at 344, an iris scanner 276 shown at 345, or the like. In embodiments, the sensors deployed in a single or multiple optical access systems, the front of which is shown in FIG. 3D. The number of visible optical access ports may vary according to the implementation of the manufacturer of the kiosk. In embodiments, and in FIG. 2B, the sensor suite for a health assessment system may comprise a glaucoma sensor 271, which may be mounted at 341 in FIG. 3D, a diabetes sensor 272, shown at 342, a macular condition sensor 273, shown at 343, a blood pressure/pulse rate sensor 274, shown at 344, an Alzheimer's sensor 275, shown at 345, or an iris scanner 276, shown at 346. In embodiments, the iris scanner 276 may be located at the top center of the user Interface in FIG. 3D, at 340. In embodiments, an access jack 347 may be incorporated to accommodate external, alternate sensors such as, but not limited to, an electronic stethoscope or a direct blood glucose measurement sensor, not shown.

In embodiments and in FIG. 3E, a single optical lens system may sense multiple characteristics of the various parts of an eye in a single image and pass the image to a number of different CCD devices for simultaneous analysis according to the principles and methods used for each type of analysis selected. Such analysis may be implemented according to image-splitting and separation methods well-known in the optical arts. In embodiments, fewer optical access modules may be used for data acquisition.

In embodiments, the systems and methods disclosed herein may comprise an assembly for a multiple sensor suite 370. The assembly may comprise an adjustable lens focusing system 371. The adjustable lens focusing system 371 may be controlled by a lens adjustment system 372. The lens adjustment system 372 may receive inputs from a suitable controller processor 352, or a separate processor 351. The assembly may receive inputs from a user's eye 361, or specifically a user's cornea 362. A beam splitter 373 may be used to separate the input received from into multiple components via partially silvered mirrors 380, via well-known techniques in the optical arts. The input is split into 2 separate beams at 382 and 383. The beams may be directed towards CCD sensors for detection and conversion into a digital image, as shown at 374 and 376. The digital image data may then be delivered to special processors 375 and 377 for analysis and rendering. The results may be delivered to the processor 351 for further manipulation, storage in RAM 353 or ROM/HD memory 354. An optional third beam may also be split via the addition of another half silvered mirror, not shown. The third beam may be directed to another CCD device at 378, and its digital image may then be delivered to a suitable processor 379 for analysis and delivery to the processor 351. In embodiments, each sensor may be configured with an internal target for a user to look at. The adjacent cameras may take images of the eye and when the user eyes fit the observational position, indicating that the user is looking at the internal target, the sensor may then be activated to perform its data acquisition process.

In an embodiment, the CCD sensors may have optical filters installed on the surface, to filter for desired wavelengths. These filters, which are located between the beam splitter output and the input to the CCD, not shown, permit detection of desired wavelengths and reduce the amplitude of out-of-band wavelengths. The choice depends of the function of the sensor. Results may be displayed to the user on display 350. The input for sensor selection function may be done via the input control device at 360.

In embodiments, a single sensor may be used for multiple functions. In embodiments, multiple functions for measuring the cornea shape and determining the eyeglasses fitting dimensions may be implemented via a three-dimensional surface scanner system. One such 3D scanner, made by DotProduct, Inc., uses structured light to illuminate a surface such as the cornea, or the face and eyes of a person. The structured light may consist of an array of very small, closely spaced circular dots or other structural elements that impinge on the surface of interest. Typically infrared light is used, and the image capture device has a filter to allow passage of infrared and exclude the rest of the light spectrum. The camera captures the image on the dots on the surface, and via various image-processing techniques involving measurement of the shape of the dot and its size, as well as the spacing between adjacent dots, determines the distance from the structured light source to each dot on the surface. The dot pattern array spacing for a flat surface is known in advance; as such, the X, Y, and Z dimensions of the surface may be calculated based on the deviation of dot locations on an actual, curved surface from the spacing that would have been present on a flat surface. With sufficiently small dot pattern spacing, the cornea can be mapped with enough precision to enable preparation of a contact lens. Similarly, the important parameters for eyeglasses fitting can be determined, such as the spacing between the two eyes, pupil to pupil, the width of the face, the location of the ears, and the like. Close-range 3D scanners are known in the high precision measurement arts for inspection of parts, and the like.

In embodiments, the user's image as captured by the 3D scanner may be displayed for real-time viewing by the user. The user may select a type of frame from a menu displayed on the user interface display screen. The selected frame design and dimensional parameters may be adjusted to fit the dimensions determined by the 3D scanner for the user's face, eye, and ear locations. The selected frame with proper dimensions can be modeled in 3 dimensions, and an image of the selected frames may be displayed to the user. In addition, an image of the frames may be overlaid on the current image of the user as captured in real-time by the scanner, via well-known augmented-reality (“AR”) techniques. In this manner, the user may see exactly how the selected frame will look on his face. The user may move his head around and see the frames from a variety of look angles, exactly as he would see it if he had real frames on while in a store. The AR technique may generate a model of the head as determined from instant samples of the user's head, face, eyes and ears, and generate anchor points for fixing the selected and dimensioned frames to the head model. These anchor points may comprise the pupil of the eye locations, top edge of ear joint to head locations, bridge of nose, or the like or any combination thereof. Once the anchor points are selected, for example, by an algorithm operating in a processor, the head model and the frame model may be joined. Location of the head as determined by the scanner may then be used to determine head position display of the joined model in the display image. Since the frame model is now anchored to the head, the anchored frame image will move with the instant image of the user's head/face, generating the effect of a virtual mirror for eyeglasses fitting.

Several systems and methods for fitting an eyeglasses frame to a user exist in the art and are commercially available. One such product is the MVTec Software GmbH/Munich HALCON software image processing for eyeglasses system. The MVTec Software allows for sample-based identification, 3D surface comparison, 3D object processing, and photometric stereo surface inspection. Other methods known to the art can be found in US 2009/0051871 “Custom Eyeglass Manufacturing Method” by Dreher et al., which analyzes multiple images captured from a digital camera in order to identify the relation of specific points of interest on a frame to outfit eye glasses. This patent application is incorporated by reference herein, in its entirety. The '871 uses an image processer to determine pupil position relative to a spectacle frame captured in front and side images. Another method for fitting an eyeglasses frame to a user is provided by U.S. Pat. No. 6,682,195 “Custom Eyeglass Manufacturing Method” by Dreher et al., which utilizes a wavefront measuring device with multiple cameras directed at a user's face and applies the measurements to a mold of a patient's head. The images are then processed by a computer to determination the location of key measurements such as pupils, center of pupils, pupil distance, width of face, ear location, distance of corneal apex from the wavefront machine, and others. These methods and systems may be incorporated into a system as described herein to provide eyeglass fitting capability and the entire disclosure of each is incorporated by reference herein, in its entirety.

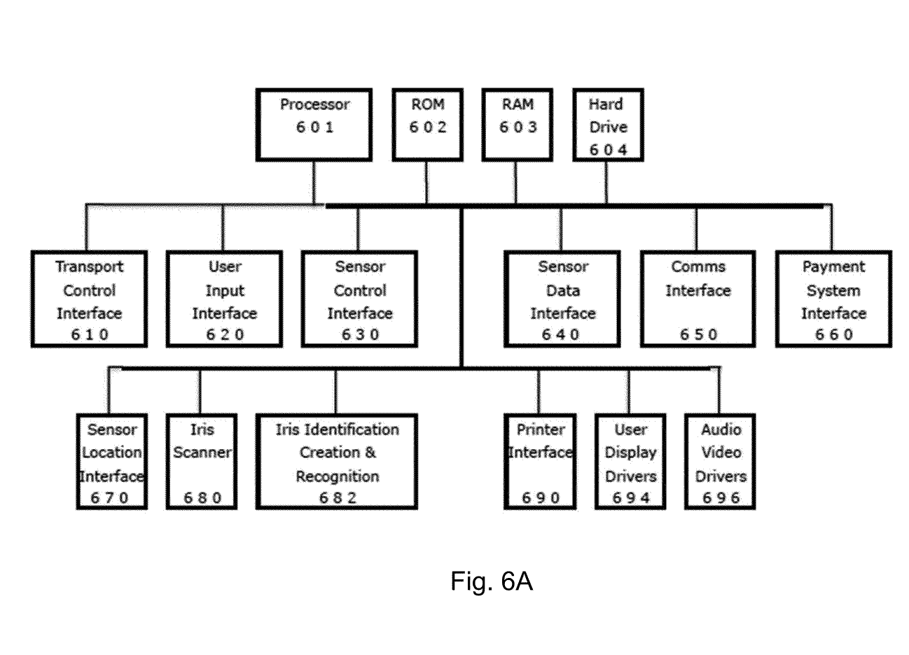

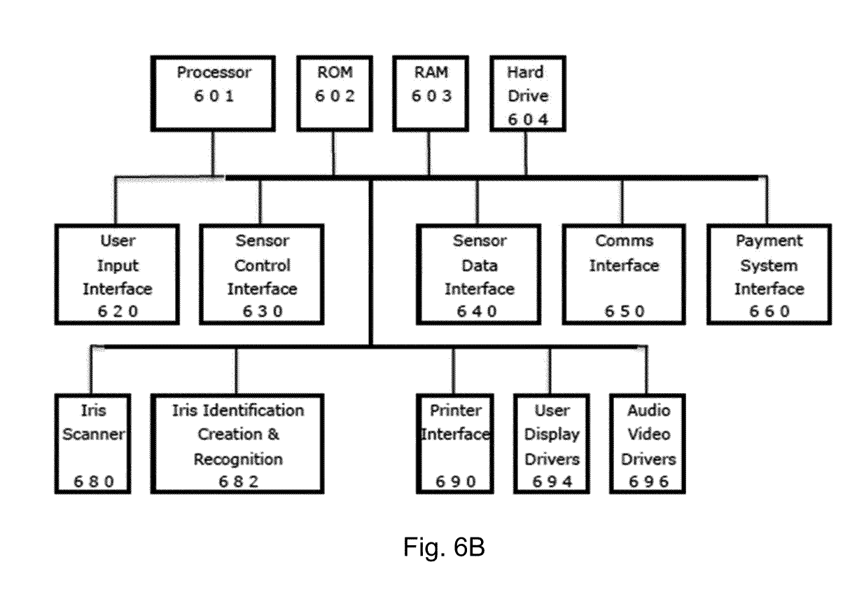

In embodiments, the 3D scanner may be used as an indicator of relative head positioning, via eye detection. The multiple centering cameras shown in FIG. 5 at 511 may be replaced by a single 3D scanner which can determine where the user's head/face is relative to a sensor that has been selected, and guidance indicators as shown at 411 in FIG. 4A can be used to help the user move his head into a proper position for the examining sensor. Often the examining sensor will have additional indicators built into the main sensor element 410 to indicate when the user has properly positioned his head. In embodiment and in FIG. 6A and FIG. 6B, the control system for the transport mechanisms may perform additional fine-adjustments to achieve the alignment needed.

The 3D scanner may be located at any of the locations cited and shown in FIG. 3B, 3C, or 3D. The only information required in order to coordinate and calibrate measurements will thus be the spatial relationships between the 3D scanner and the various other sensor systems. In embodiments, the 3D scanner may be a separate module installed in the carousel system as well.

In embodiments the assessment systems may be integrated with a plurality of subsystems. For example, in embodiments, a contact lens prescription generator may be supplied, along with a corneal mapping unit, to provide both the correction prescription and a surface map of the cornea to aid the making of a contact lens that fits the user properly. The original map may be reduced to a mathematical model via various methods shown in the literature. Such mathematical models may enable a custom-made lens to be produced on a mold made from the mathematical model. Optionally, an eyeglass prescription generator may be included, or may be offered independently in a separate stand-alone system. Optionally, an eyeglass frame fitting analyzer may be included with the eyeglass prescription generator. Options for frame choice may be shown on a user display. In embodiments, the system may generate prescriptions for bifocals, trifocals, continuously variable corrections, sunglasses, and the like. Measurements of body parameters for eyeglasses fittings may be obtained from images of the user's face and head, taken by cameras that may also be used for sensor alignment to the user's eyes. Examples of the functionality of the prescription generator may include, but is not limited to, dispensing a printed prescription for the user or forwarding the prescription directly to a contact lens provider. The user may make a payment for all services via a credit card reader system or other payment system known to the art, as installed in the main housing. One such payment system is the VenTek International Corp., which may provide PCI (Payment Card Industry) certified payment systems with automated pay stations and PCI PA-DSS (Payment Application Data Security Standard) compliant revenue collection from the vision and health assessment systems disclosed herein. Payment Cards may include both credit cards and other kinds of cards as may be issued by insurance companies, such as those which provide coverage for health assessment screening and/or vision prescription and other forms of vision care. In addition to various embodiments of point of sale payment systems, which are incorporated herein by reference in their entirety, payment system management programs may also be incorporated. One such management system is the venVUE, which is a web-based platform. It provides real-time pay station status, remote active and passive monitoring, remote pay station configuration and report generation for status, operational statistics, revenue collection and reconciliation.

In embodiments, the sensor suite may comprise a contact lens auto-refractor. The auto-refractor may be any one of the several auto-refractors known to the art and commercially available, such as, but not limited to, The Canon RK-F1 Full Auto Refractor-Keratometer, the Marco Nidek ARK 530A Auto Refractor Keratometer, the Tomey RT-7000 Auto Refractor/Topographer, the Right Medical Speedy-K Auto Refractor Keratometer, and the like. Several eyeglasses auto-refractors are also known to the art. Once such auto-refactor is the CHAROPS CRK7000 Autorefractor/Keratometer. The CRK7000 uses two mire rings and two LEDs to provide corneal curvature radius and corneal refractive power.

In embodiments, the sensor suite may comprise corneal topography and imaging sensors in order to examine a user's corneas to verify that the user may be fitted with a contact lens. The corneal topography and imaging sensor may be one of the several technologies known to the art and commercially available. In embodiments, the sensor suite may include a sensor such as, but not limited to, the Scout Keratron Corneal Topographer, from Eyequip, which is a portable topographer for corneal topography and imaging. The Scout Keratron may be adapted to function as an affixed component of a sensor suite. Alternatively, in embodiments, the sensor suite may comprise the Orbscan Hz Corneal Analysis System by Bausch & Lomb, which is a multidimensional diagnostic topography system that maps and analyzes elevation and curvature measurements of the entire anterior and posterior surfaces of the cornea. The Orbscan II corneal topography system may also perform pachymetry measurements to determine the thickness of the cornea in a non-contacting method. Such a method may be useful for glaucoma testing. In other embodiments, the sensor suite may comprise the Pentacam, manufactured by Oculus, which is a combined device using a slit illumination system and a Scheimpflug camera which rotates around the eye. In embodiments, the systems and methods disclosed herein may comprise software configured to assess the health of the cornea and the likelihood that a user's eye(s) may be fitted with a suitable contact lens. This software is designed to detect anomalies in the shape or surface or interior of the cornea. Once the cornea is judged to be able to accept a contact lens, the auto-refractor can determine the proper correction prescription. A prescription can be written for any kind of contact lens, including the two most popular types: rigid gas permeable (hard) and silicone hydrogels (soft).

In embodiments, a specialized sensor may be employed to measure and map the cornea so that the anterior surface of a contact lens can be custom-made to fit each eye. For example, a three dimensional scanners such as the above mentioned DotProduct, though not limited to such products, may be used to measure and map a cornea for contact lens customization.

In embodiments, a specialized sensor may be employed for diabetes testing. There are several products and methods known to the art and commercially available for diabetes testing. One such product is the CLEARPATH DS-120, a non-invasive instrument for measuring the level of blood glucose in the fluids and structures of the eye. The product recently was approved by the Food and Drug Administration of the U.S. Government with a 510(k) clearance and is available from Freedom Meditech, Inc. It identifies levels of elevated Advanced Glycosoloated End Products [“AGEs”] by measuring the intensity of fluorescence and scattering of light in the lens during a brief scan. In embodiments, other sensors to determine glucose non-invasively may be used. One such sensor is the Dione bidirectional sensor, produced by Lein Applied Diagnostics. The Dione is a bidirectional sensor which is a compact and affordable source/detector module that can be used as a scanning or static device, using confocal micrology technology. The Lein technology may be used to determine distance and position, thickness, as well as refractive power.

In embodiments, a specialized sensor may be employed for blood pressure and pulse rate detection. Such methods are readily known to the art. One such method is provided by Poh et al. in their article “Non-contact, automated cardiac pulse measurements using video imaging and blind source separation,” printed in Optics Express, Vol. 18, Issue 10, pp. 10762-10774 (2010). Poh et al. used automatic face tracking along with blind source separation of color channels into independent components, then used Bland-Altman and correlation analysis to correctly predict physiological readings from a user's facial movements.

In embodiments, a sensor may be employed to determine macular condition of a user's retina. Such sensors and methods are readily known to the art. One such sensor is the SECO International Inc. EasyScan zero-dilation retina. This camera may provide an image of the retina based on Scanning Laser Opthalmoscope technology (“SLO”). SLO uses horizontal and vertical scanning mirrors to scan a specific region of the retina and create raster images that may be viewable.

In embodiments, sensors may be employed to determine retinal circulatory physiology. Such sensors and methods are known the art. The EasyScan and the abovementioned CLEARPATH DS-120 may also be used to image blood vessels in the retina. Analysis of these images by a suitable program can identify abnormalities in the size and shape of the blood vessels, which may indicate existence of a number of physiological readings, such as hypertension.

In embodiments, the health assessment system may be configured to provide additional testing subsystems as described herein. These additional testing subsystems may be added any configuration for vision screening, supplied individually, or used in combination with other subsystems.

In embodiments, eye examination sensors for the various assessment subsystems may each be mounted on a carousel wheel that may be rotated to present a particular assessment subsystem sensor to the face of the user. The wheel may be located behind a viewing panel that opens to the user when activated. The particular assessment subsystem senor is extended from the wheel so that it may be brought closer to the user's face, and available directly to the user's eyes. In another embodiment, the sensors may be stored in a file-cabinet like structure, and conveyed from that structure to the front of the housing, where the sensor may be positioned by automatic sensing of the user's face and eye locations. Other embodiments for storage and retrieval are also feasible.

Additional details of the mounting and transport systems 111 and 211 in FIG. 1A and FIG. 2A respectively are shown in FIGS. 4A, 4B, and 5. In embodiments and in FIG. 4A, the systems and methods disclosed herein may comprise a mounting and transport system for conveying various sensors to the examination window and in proximity to the user's eyes. The overall system is referred to as the Visual System User Interface Assembly 400. The system may comprise a wheel box with the sensor suite contained therein, at 401. The system may further comprise a height adjuster 402 to bring the selected sensor into alignment with the location of the user's eyes. A depiction of a user's head is shown at 413 and 414, showing different height elevations for different users. Elevation may be controlled by the control system 155 for the prescription system of FIG. 1A or 255 for the health assessment system of FIG. 2A. The height adjustment may be accomplished manually by user input to the user input system 120 or 220. It may also be accomplished automatically by use of cameras and software configured to look for eye shapes as the elevation adjust 402 moves upwards from a rest position. The range of vertical motion of the wheel box 401 is shown at 403. The wheel box 401 comprises a rotating platter on which various sensors are affixed as shown at 407 and 409. In embodiments the system may comprise multiple sensors 408. The system may comprise is a main sensor 410 for performing the analysis. The system may comprise two cameras at 409, camera 1 and camera 2, which may be used to locate the user's eyes and create a feedback system of indicators around the periphery of the main sensor 410 to indicate when the user has positioned his eye in the proper place. Such eye alignment sensors 411 may be incorporated with each separate testing sensor in the sensor suite 407 as may be desired or necessary. When the desired sensor for a particular function is selected, the wheel may rotate to bring the selected sensor into proper position at the examination window 405. The sensor system may be extended from its location on the wheel, to bring the sensor into closer proximity with the user's eye[s] as shown at 412. In embodiments, the system may comprise a head supporting system, which may extend from the face of the housing in a recessed position or in an extended position 406, permitting the user to rest their chin on the support system, and get into proper position for a chosen sensor to examine their eyes. When the chin rest system is employed, the height elevation system may be activated first, in order to bring the chin rest to a comfortable position for the user.

In embodiments, upon completion of the examination, the head support system and sensor face may undergo a cleaning step. Additionally, the sensor may include a protective transparent cover located between the sensor and the user. An automated dispenser of an antiseptic fluid that leaves no residue may be applied to the front surfaces of the sensor, eliminating a need for the sensor to be wiped off. In embodiments, a user may receive feedback at various steps in the analysis, indicating whether further testing is feasible, or if a prescription can be generated.

In an embodiment and in FIG. 4B, the systems and methods disclosed herein may comprise an alternate transport system for the sensor suite 170, 270, the file cabinet storage system 419. In embodiments, the wheel/carousel 404 and the rotation mechanism may be replaced with a file-cabinet style of sensor module storage 420. In embodiments, the storage system may comprise multiple sensor modules 421, 422, 423. A first module 422 may be extracted from the cabinet storage 420 by the sensor transport system 424, and brought into close proximity to the examination window aperture 425. The sensor transport system may use linear motors to move the sensor modules from the storage location 420 to the user examination location 426. Such transport mechanisms are well known in the mechanical arts in such items as soft-drink dispensers and other vending machines. In embodiments, the transport mechanism 424 may be configured to move in two dimensions, vertically and horizontally, in order to access and retrieve and place sensor modules according to a control scheme 155, 255 implemented in FIGS. 1A and 2A.

Alternate storage cabinet embodiments 420a, 420b, 420c are shown in FIG. 4B. The modules may be stored vertically 420a, horizontally 420c, or in combination 428. In embodiments, different storage patterns and organization may be implemented depending on a number of factors, such as, but not limited to, space restriction, number of modules, type of modules, and the like.

In embodiments and in FIG. 4B, an optional chin rest and face alignment system 430 may be implemented at the examination window as an aid to proper positioning of the user's head and eyes.

In embodiments and in FIG. 5, the systems and methods disclosed herein may comprise a secondary transport and positioning mechanism suitable for operation with either the carousel/wheel sensor suite access system or a file-cabinet style of sensor module storage is depicted. In this configuration, the sensor module may be first retrieved from a storage position if stored in a file-cabinet storage facility, or the carousel is rotated to bring the selected sensor into juxtaposition with the examination window. When used in conjunction with a carousel, the sensor module 522 may be extended from its resting place on the carousel and brought in closer proximity to the user's head, via the examination window 525. In this embodiment, the examination window 525 may be part of a Face Alignment Transport Mechanism 540, which brings the examination window 525 and the sensor module 522 and the sensor transport mechanism 524 into closer proximity to the user's head and eyes. The Face Alignment Transport Mechanism 540 passes through an aperture 541 in the front face of the user interface surface 542. When used with the file-cabinet storage facility, the sensor transport mechanism 524 is the same as shown in FIG. 4B at 424. In an embodiment and in FIG. 5, the sensor module may be moved transversely 530 to the examination window, left or right, to align the sensor with either the left eye or the right eye, shown at 531 and 532 respectively. In embodiments, the system may also comprise eye-centering cameras 511, 411. In embodiments, the system may comprise Linear motor transport drivers 550, 551, 552 which may operate in conjunction with the sensor transport system 524, the Face Alignment Transport Mechanism 540, and the left-right positioning mechanism 530, respectively. A fourth linear motor 553 may also be used to control the elevation of the sensor 533 which may be located in the sensor module 522. These motors may be controlled by the control system 155, 255. In embodiments, the system may comprise a chin rest 545 below the examination window 525.

The health assessment system may be configured for user access while either sitting or standing. When sitting, a chair may be free standing or affixed to the system via a connecting platform, and have an adjustment for height and separation distance from the front face of the enclosure.

Sources for each sensor may be customized, modified from a third party, used off-the-shelf, and the like. Sensors may include, but are not limited to, a contact lens auto-refractor, a corneal mapping sensor, an eye glasses auto refractor, an eye glasses frame fitting sensor, a glaucoma testing sensor (such as via pachymetry), a diabetes testing sensor, a sensor for evaluating the macular condition of the retina, a sensor for evaluating retinal circulatory physiology, a sensor for detecting Alzheimer's disease, an iris scanning sensor, and the like. Sensors may be configured with internal targets for the user to look at. The adjacent cameras may take images of the eye and when the user's eyes are in the proper range and fit the desired observational position, indicating the user is looking at the internal target, the sensor may be activated to perform its data acquisition process.

The systems and methods disclosed herein may provide for corneal topography and imaging, such as before any auto-refracting is done, where the user's corneas are examined and where it is verified that they can be fitted with a contact lens. Software may be configured to assess the health of the cornea and the likelihood that the user's eye(s) can be fitted with a suitable contact lens. This software may be designed to detect anomalies in the shape or surface or interior of the cornea. Once the cornea is judged to be able to accept a contact lens, the auto-refractor can determine the proper correction prescription. A prescription may be written for a variety of different kinds of contact lens, such as including the two most popular types: rigid gas permeable (hard) and silicone hydrogels (soft).

The systems and methods disclosed herein may provide for corneal mapping for the contact lens' anterior surface, where a corneal spline generator may utilize a spline surface algorithm for reconstruction of the corneal topography from a video keratographic reflection pattern, or an iteratively re-weighted bi-cubic spline representation of corneal topography. Using a comparison to standard models, corneal shaping for adequate tear flow and hydration, and the like may be implemented. Additional software may be used to modify the first estimate of the corneal shape and spline rendering, to accommodate the need for proper tear flow and eliminate any pockets that would preclude irrigation of the cornea by tears. A corneal topography system may also perform pachymetry measurements to determine the thickness of the cornea in a non-contacting method.

Several corneal mapping sensors and methods are available to measure the anterior surface of a user's cornea for several purposes, such as for contact lens prescription. One such method is detailed by Mark A. Halstead et al. in their paper “A Spline Surface Algorithm for Reconstruction of Corneal Topography from a Videokeratographic Reflection Pattern.” Halstead's method uses an iterative algorithm in order to output a piecewise polynomial description of a simulated corneal surface in order to recover the three dimensional shape of a cornea from a videokeratograph image. Another method known to the art is by Zhu et al., detailed in their paper “Iteratively re-weighted bi-cubic spline representation of corneal topography and its comparison to the standard models.” Zhu's method represents the corneal anterior surface using radius and height data taken from a TMS-2N topographic system and simulates visual performance using a general quadratic function, a higher order Taylor polynomial approach, and an iteratively re-weighted bi-cubic spline method.” Similarly, U.S. Pat. No. 5,452,031 “Contact lens and a method for manufacturing contact lens” teaches a method of manufacturing a contact lens using computer implemented spline approximation of corneal topology. This method uses piecewise polynomials in order to generate a smooth measuring surface. Additional software may be used to modify the first estimate of the corneal shape and spline rendering, to accommodate the need for proper tear flow and eliminate any pockets which would preclude irrigation of the cornea by tears

The systems and methods disclosed herein may provide for the detection of diabetes, such as by measuring the level of blood glucose in the fluids and structures of the eye by identifying levels of elevated advanced glycosoloated end products (AGEs) by measuring the intensity of fluorescence and scattering of light in the lens during a brief scan.

The systems and methods disclosed herein may provide for the determining of the macular condition of the retina, such as with a zero-dilation retina camera providing an image of the retina, thereby allowing for an analysis of the macular condition of the retina. This technique may also be used to test for diabetes and for glaucoma.

The systems and methods disclosed herein may provide for a determining of retinal circulatory physiology, such as with a retina camera used to image the blood vessels in the retina. Analysis of these images by a suitable program may identify abnormalities in the size and shape of the blood vessels, which may indicate existence of hypertension, i.e., high blood pressure.

The systems and methods disclosed herein may provide for detection of Alzheimer's disease, such as through a system for detecting the presence of a polypeptide aggregate or protein in the cortical and/or supranuclear regions of a person's lens has been shown to be a precursor indicator for Alzheimer's disease. Such a system is disclosed by Goldstein et. al in U.S. Pat. No. 7,653,428 “Method for diagnosing a neurodegenerative condition,” which is incorporated by reference in its entirety herein. Other detection means may include examining retinal nerve cells undergoing apoptosis (a genetically regulated process leading to the death of cells) via imaging of the retina where the cells are marked with florescent markers, measuring the widths of retinal blood vessels (e g Alzheimer's patients show larger retinal blood vessels than in patients without the disease), and the like, where the system may be used to create an image of the user's retina, and sent to a specialist via the internet for examination. Such an approach is known to the art and has been detailed by Cordeiro et al. in their article “Imaging multiple phases of neurodegeneration: a novel approach to assessing cell death in vivo,” found in Cell Death and Disease (2010).

In embodiments, the systems and methods disclosed herein may comprise a payment system, such as a certified payment system with automated pay stations (e.g. certified PCI PADSS (payment application data security standard) compliant for revenue collection from the vision and health assessment systems. Payment cards may include, but are not limited to, credit cards and cards from insurance companies that provide coverage for health assessment screening and/or vision prescription and other forms of vision care. In addition to point of sale payment systems which are incorporated into embodiments, the system may provide for a web-based platform that provides real-time pay station status, remote active and passive monitoring, remote pay station configuration and report generation for status, operational statistics, revenue collection and reconciliation, and the like.

In embodiments, a user may activate the system by pressing a start button. The system may initiate a user Identification Process that requests the user's name and other related data. The system may initiate a request for the user to place their head in a pre-determined position in front of the system housing at the user aperture to enable an iris scan to be completed. A menu of user selectable options for service may appear on a screen. User makes a selection, and is prompted to make a payment, such as if one is required for the selected service. Some services may be free, but for those that are not, a user selects a payment option, and makes the payment. The system may execute the payment function and validate it as being paid, where the system may display a payment acknowledgement. The system may initiate the selected service and activate the sensor that can provide the selected service. The system may display instructions to the user for receiving the selected service. In embodiments, the system may either complete the service in a satisfactory manner, or not. If not, service may be cancelled and payment is refunded, or credited to another service selection that can then be made by the user. If completed, then system may provide the user with a visual display of the results of the service, and optionally prints a paper copy. The system may prepare a summary report for transmission via the communications system to a remote data storage facility. Third-party service providers may access said summary report and prepare a suitable appliance for the user, based on the report prescription. Such third-party service providers may be selected by the user or by the system by a prearranged agreement with third parties.

In embodiments, there may be a monitor and control system that monitors all inputs from all subsystems, enables the control system to make decisions based on these inputs, where the control system issues commands or initiates other outputs to various subsystems. In addition, the control system may manage the mounting and transport system which in turn controls the selection and delivery and return of the sensor subsystem needed to perform the selected service by the user, along with fine positioning control for proper sensor alignment with user's eye. The monitor and control system may also manage the Iris scan data collection for creating a user identification code, or recognizing a returning user. The monitor and control system may activate the mounting and transport system to:
i. select a sensor module based on a user selection of a service;
ii. initiate transport of sensor from sensor module storage facility to baseline examination location;
iii. activate the sensor module fine positioning process;
iv. activate sensor module or sensor movement about baseline location in response to eye imaging location data;
v. activate sensor data acquisition by command to the associated dedicated processor’ mating operational subsystem;
vi. receive indication of completion of data acquisition from said mating operational subsystem;
vii. initiate transport of sensor module from baseline examination location to sensor module storage facility;
viii. initiate a report for display to a user about test results; and initiate transmission of sensor data acquired from sensor to a remote storage facility.

Alternatively, in the case where the sensors are mounted directly on the surface of the housing that comprises the user interface, each sensor may be activated directly, and an indicator light may be employed to direct the user to put his face near the operative sensor.

The monitor and control system may include a processor configured to execute commands and operations based on a stored program, stored in a memory; digital storage components comprising read only memory (ROM), random access memory (RAM), and a hard drive; an iris scanner; a stored program for creating a user Identification code based on data from the iris scanner; a stored program for accessing a secure database with an iris scan of a returning user to seek a match with the user's stored Identification code, and the like. A group of interfaces may include transport control, user input, sensor control, sensor data, communications, payment systems, a printer, a user display, an audio-video display, and the like.

The mounting and transport system may manage the selection of an appropriate sensor module for the test or service to be provided, as determined by the user input. The mounting and transport system may accesses a database for the location of the appropriate sensor module for the selected service, activates a transport mechanism that selects the desired sensor module, and then commands the transport mechanism to bring the sensor module to a baseline examination location. This location is near the user service aperture. A second positioning system receives commands from a fine positioning controller to maneuver the sensor module into a suitable location for accessing an eye of the user. The fine positioning system receives commands from the monitor/control system based on inputs to the eye location and cameras or other appropriate sensors. This positioning system may move the sensor module in two or three directions, according to embodiments of the systems and methods disclosed herein. The sensor module may be positioned at a location extending thru the user aperture, or from within it.

The components of the transport system may include a microprocessor for controlling various motors and drives; a sensor module receptacle device for holding a sensor module; a conveyor system for moving a sensor module from a storage facility to a baseline examination location; a fine positioning system for adjusting the position of the sensor module about the baseline examination location, in either two or three orthogonal directions; a plurality of linear drive motors for moving the sensor module receptacle device and for moving the sensor module, and for moving the sensor itself, and the like. The linear drive motors may comprise the conveyor system directly. Such a drive motor system may comprise a motor attached to a gear, which in turn engages a linear gear affixed to the base. Location detection sensors for creating location information about the location of the sensor module for use by the transport control system and the monitor and control system.

The payment system may include a number of subsystems, integrated with the overall vision and health assessment system. The payment system is configured to perform a plurality of tasks, such as accept a credit card for payment; perform validation of the card; effect a transfer of funds from the credit card account to another account; create a receipt for the user; create a record of the transaction per normal credit card activities; create a database entry for the user associated with the user ID created by the iris scanner; inform the monitor and control system that payment has been made and the selected service may be performed; accept cash as a form of payment and perform the previous steps as appropriate; and the like. The payment system may include a card reader; a microprocessor based control system; a data management and data processing program; and the like.

In embodiments, the systems and methods disclosed herein may include a communications system, such as providing the system with access to the Internet via a variety of alternative methods for reaching a point of presence, where many of the transactions involving remote parties may be undertaken via Internet access. Management information regarding sales and service activities, payments, customer identification, orders for filling prescriptions for contact lenses, eyeglasses, and frames may be transferred from an assessment system to the appropriate providers, such as via direct transmission to any provider, via data storage in a remote secure facility, and the like, which may then be accessed by authorized prescription providers. The communications system may include a data formatter for accepting data from the monitor and control system, as received from a sensor subsystem; a modem for creating or decoding a suitable packet data transmission; a data communications system for accessing the internet; a connection to an internet service, such as at a point of presence; and the like. The connection to the Internet may be by a wireless device or may be wired directly via a telephone service or a cable TV service.

In embodiments and in FIG. 1A, the systems and methods disclosed herein may comprise a vision correction system for determining a prescription for vision correction for a user in a semi-automated system. The system may include a housing; a processor; a user interface 101 for interacting with the vision correction prescription system; a user identification subsystem; at least one sensor associated with a mating operational subsystem 170i for examining a user's eye; a mounting/transport system 111 for said sensor; a monitor system 150 for receiving and processing data from said sensor and mating operational subsystem for said user's eye; a control system 155 for operating said mounting system and said sensor; a prescription generator system for creating a prescription for a vision correction appliance based on processed data from said sensor and mating operation subsystem; a communications link 165 connected to said monitor system, and the like. In embodiments, the user interface may include a user optical interface for interacting with said health assessment sensor subsystems 110; a user selection system for selecting a health assessment subsystem user input 120; a user results display system 130; a printer for printing user information 139; a user payment interface 140, and the like. The user optical interface 110 may further include an aperture in said housing; a facial engagement system for aligning the user's head with said aperture; a head location system for determining positioning information about the user's head location; an iris scanning system 176 for creating or assessing a user ID; and the like. The user selection system may further include a display screen for displaying information to the user; a keyboard for choosing a service option from the system; an audio system for supplying additional aural information; and the like. The user results display system may include a visual display connected to the control system. The printer may be connected to the control system. The user payment interface may further include a keypad and a display; a credit card reader; a processor configured to accept a credit card number from a credit card inserted in said reader and perform an online banking transfer for payment of services from said credit card to another bank account; and wherein said processor is connected to said control system, and the like. The online banking transfer may be performed via an Internet connection. The sensor associated with a mating operational subsystem may include a contact lens auto-refractor subsystem 171. The sensor associated with a mating operational subsystem may include a corneal mapper subsystem 172. The sensor associated with a mating operational subsystem may include an auto-refractor for eyeglasses subsystem 173. The sensor associated with a mating operational subsystem may include a glasses frame fitter subsystem 174.

In embodiments, the systems and methods disclosed herein may provide for a mounting/transport system 111 for the sensor may include a sensor module for housing said sensor; a storage facility for a plurality of sensor modules; a transport system for conveying said sensor module between the storage facility and a baseline examination location; and the like. The mounting/transport system may include a cleaning system for sanitizing the sensor after each use, wherein the cleaning system is at least one of an alcohol sprayer and a compressed air blast to blow away loose material on the sensor.

The storage facility may include a circular platform. The sensor modules may be located on radials of the circular platform. The storage facility may include a plurality of compartments, such as arranged in a planar configuration accessible by said transport system (e.g. FileCabinet Style). The planar configuration may have a vertical access face. The transport/transport system may further include a transport mechanism for moving the sensor module about said baseline analysis location in any of three dimensions: in-out, left-right, and/or up-down. The transport system may further include a transport mechanism for moving the sensor module from said baseline analysis location in any of two dimensions, e.g. vertically or horizontally, in/out or up/down. The sensor module may further include a plurality of cameras located around the periphery of the sensor module to provide at least one image of a user's eye region. The cameras may be connected to the control system to provide an image of a user's eye region, to a corresponding dedicated processor remote from the sensor module. The sensor system and said transport system may be operated by the control system. In embodiments, the communications link may include a direct wired connection to a point of presence for internet access; a direct wired connection to a point of presence on a cable system; a wireless terrestrial connection to a point of presence for internet access; a satellite wireless connection to a point of presence for internet access; and the like. The data from the sensor for the user's eye may be associated with a user identification code created by said user identification system in a user data set. The user data set may be stored via said communications link in a remote database.

In embodiments and in FIG. 3B, the sensors may also be mounted 327 to a facet of the housing. In this embodiment, the sensors may be activated by the control system and the user places his face and eyes in front of the active sensors 340. In embodiments and FIGS. 6A and 2B, the user interface 327 may include a touch screen display for showing the user the results of any analysis, and provide for softkey inputs to the control system 620, 230. In embodiments and in FIGS. 3B, 3C and 3D, the user input mechanism may include a computer keyboard 360. In embodiment, the sensor suite may be mounted on the user interface 327, eliminating the need to activate a transport mechanism.

In embodiments, the systems and methods disclosed herein may provide a monitor system 250 including a memory system; a bus; a processor configured to execute steps from a program stored in the memory system, wherein said processor initiates functions comprising display options and menu choices; receive inputs from said user selection system; activate a health assessment sensor in response to a user selection; monitor user head position; receive and analyze data from head location system; monitor user eye position; receive and analyze data from eye cameras; provide feedback to user regarding said head or eye position; activate an iris scan system for user identification purposes; receive status information from said selected sensor system; receive health assessment information from said selected sensor system; provide results in a user-friendly format to said user results display system; and the like.

In embodiments, the systems and methods disclosed herein may provide a control system 255 including a memory system; a bus; a processor configured to execute steps from a program stored in said memory system, in response to inputs from said monitoring system, and inputs from status indicators associated with said sensor with a mating operational subsystem, wherein said processor initiates functions comprising: select a sensor module; initiate transport of sensor from sensor module storage facility to baseline examination location; activate sensor fine positioning process; activate movement about baseline location in response to eye imaging location data; activate sensor data acquisition; receive indication of completion of data acquisition from said mating operational subsystem; initiate transport of sensor module from baseline examination location to sensor module storage facility; initiate transmission of sensor data acquired from sensor to a remote storage facility; and the like. In embodiments, the present systems and methods disclosed herein may include a housing, such as in the form of a kiosk configured for user access while the user is standing, as a stand-alone module configured for user access while sitting, and the like. In embodiments, the systems and methods disclosed herein may include a user identification system comprising a data entry system coupled to said monitoring system; an iris scanning system coupled to said monitoring system; an encryption system for encoding data from said data entry system representative of a user's identity with data from said iris scanning system, producing a user identification code; and the like.

In embodiments, a specialized sensor may be employed for iris scanning Several iris scanning systems and methods are known to the art. One such system is disclosed by Daugman in U.S. Pat. No. 5,291,560 “Biometric personal identification system based on iris analysis,” which is incorporated by reference in its entirety herein. The systems and methods disclosed by Daugman can scan an iris using image analysis algorithms to find the iris in a live image of a user's face, then encode the texture into a compact signature. The texture is then extracted from the image by a self-similar set of quadrature bandpass filters defined in a dimensionless polar coordinate system. The sign of the projection of the many different parts of the iris onto the multi-scale quadrature filters then determines each bit in an iris signature. Such a sensor may work the monitor and control system as well as the payment system.

In embodiments, the systems and methods disclosed herein may provide for a prescription generator that may include a stored program for execution on configured to receive a data set from an auto-refractor sensor and its mating subsystem; determine a correction prescription for a contact lens to correct the data set to within a specified level of correction; provide said correction prescription to said user interface; and the like. The prescription generator may include a stored program operative on said processor and configured to receive a data set of a corneal scan for a user's eye; determine a best-fit model for the front surface of said cornea; compensate said best-fit model to accommodate tear flow and minimize potential voids; and provide said corneal map for creating the anterior portion of a contact lens to said user interface. The prescription generator may include a stored program for execution on configured to receive a data set from an auto-refractor sensor and its mating subsystem; determine a correction prescription for a pair of eyeglasses to correct the data set to within a specified level of correction; provide said correction prescription to said user interface; and the like. The prescription may be selected from a single prescription; bifocal prescription; trifocal prescription, continuously variable correction prescription; and the like. The prescription generator may include a stored program operative on the processor and configured to receive a plurality of images from at least one camera controlled by said control system wherein said images provide a digital image of the user's eyes, face and side of head; processing said digital image to derive a proposed size and shape of eyeglasses lenses, an estimate of the inter-ocular distance between the user's eyes, and the size of temples for the eyeglasses; and provide said prescription for eyeglasses frames and lens size to the user interface.

In embodiments, the systems and methods disclosed herein may include a health assessment system for providing health assessment of a user's health condition in a semi-automated system, comprising a housing; a user interface 201 for interacting with the health assessment system; a user identification subsystem; at least one sensor associated with a mating operational subsystem 270i for examining an eye; a mounting/transport system 211 for said sensor; a monitor system 250 for receiving and processing data from said sensor and mating operational subsystem; a control system 255 for operating said mounting system and said sensor; a communications link 265 connected to said monitor system; and the like. In embodiments, the user interface may include a user optical interface for interacting with said health assessment sensor subsystems 210; a user selection system for selecting a health assessment subsystem user input 220; a user results display system 230; a printer for printing user information 239; a user payment interface 240; and the like. The optical interface 210 may include an aperture in said housing; a facial engagement system for aligning the user's head with said aperture; a head location system for determining positioning information about the user's head location; an iris scanning system for creating or assessing a user ID; and the like. The user selection system may include a display screen for displaying information to the user; a keyboard for choosing a service option from the system; an audio system for supplying additional aural information; and the like. The user results display system may include a visual display connected to the control system. The printer may be connected to the control system. The user payment interface may include a keypad and a display; a credit card reader; a processor configured to accept a credit card number from a credit card inserted in said reader and perform an online banking transfer for payment of services from said credit card to another bank account; and wherein said processor is connected to said control system; and the like. The online banking transfer may be performed via an Internet connection. The sensor associated with a mating operational subsystem may include a glaucoma testing subsystem, a diabetes testing subsystem, a macular examination subsystem, a retinal circulatory physiology examination subsystem, an eye lens analysis subsystem for assessing the presence of Alzheimer's Disease; and the like.

In embodiments, the systems and methods disclosed herein may provide for a mounting/transport system 211 including a sensor module for housing said sensor; a storage facility for a plurality of sensor modules; a transport system for conveying said sensor module between the storage facility and a baseline examination location; and the like. The storage facility may include a circular platform. The sensor modules may be located on radials of the circular platform. The storage facility may include a plurality of compartments, where the plurality of compartments may be arranged in a planar configuration accessible by said transport system (e.g. FileCabinet Style). The planar configuration may have a vertical access face. The transport/transport system may include a transport mechanism for moving the sensor module about said baseline analysis location in any of three dimensions (e.g. in/out, left/right, or up/down). The transport system may include a transport mechanism for moving the sensor module from the baseline analysis location in any of two dimensions, e.g. vertically or horizontally, in/out or up/down. The sensor module may include a plurality of cameras located around the periphery of the sensor module to provide at least one image of a user's eye region. The cameras may be connected to the control system to provide an image of a user's eye region. The sensor module may be connected to a corresponding dedicated processor remote from the sensor module. The sensor system and the transport system may be operated by the control system.

In embodiments, the present systems and methods disclosed herein may provide for a monitor system including a memory system; a bus; a processor configured to execute steps from a program stored in said memory system, wherein the processor initiates functions comprising: display options and menu choices; receive inputs from said user selection system; activate a health assessment sensor in response to a user selection; monitor user head position; receive and analyze data from head location system; monitor user eye position; receive and analyze data from eye cameras; provide feedback to user regarding said head or eye position; activate an iris scan system for user identification purposes; receive status information from said selected sensor system; receive health assessment information from said selected sensor system; and provide results in a user-friendly format to said user results display system. The control system 255 may include a memory system; a bus; a processor configured to execute steps from a program stored in said memory system, in response to inputs from said monitoring system, and inputs from status indicators associated with said sensor with a mating operational subsystem, wherein the processor initiates functions comprising: select a sensor module; initiate transport of sensor from sensor module storage facility to baseline examination location; activate sensor fine positioning process; activate movement about baseline location in response to eye imaging location data; activate sensor data acquisition; receive indication of completion of data acquisition from said mating operational subsystem; initiate transport of sensor module from baseline examination location to sensor module storage facility; initiate transmission of sensor data acquired from sensor to a remote storage facility; and the like. The housing may take the form of a kiosk configured for user access while the user is standing, a stand-alone module configured for user access while sitting, and the like. The communications link may be provided including a direct wired connection to a point of presence for internet access; a direct wired connection to a point of presence on a cable system; a wireless terrestrial connection to a point of presence for internet access; a satellite wireless connection to a point of presence for internet access; and the like. The data from the sensor for the user's eye may be associated with a user identification code created by the user identification system in a user data set. The user data set may be stored via the communications link in a remote database. The user identification system may include a data entry system coupled to said monitoring system; an iris scanning system coupled to said monitoring system; an encryption system for encoding data from said data entry system representative of a user's identity with data from said iris scanning system, producing a user identification code; and the like. The mounting/transport system may include a cleaning system for sanitizing the sensor after each use, wherein the cleaning system include an alcohol sprayer, a compressed air blast to blow away loose material on the sensor, and the like.

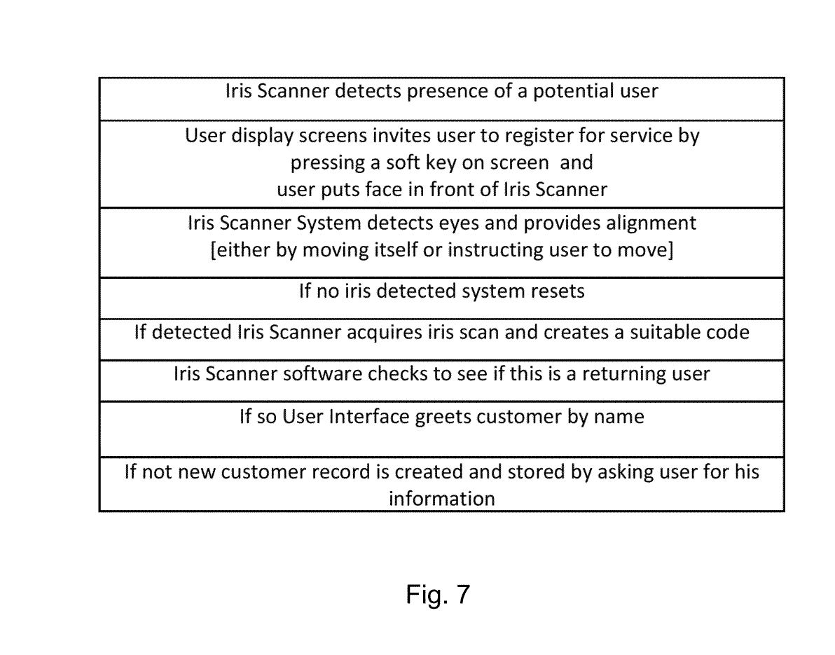

In embodiments and depicted in FIG. 7, the systems and methods disclosed herein may first comprise using the iris scanner to detect the presence of a potential user. The user display screen then invites user to register for service by pressing a soft key on screen and a user puts his or her face in front of the iris scanner. The iris scanner system may then detect the user's eyes and provide alignment, either by moving itself or instructing a user to move. The iris scanner then may acquire an iris scan and create a suitable code. If no iris is detected, the system may reset. The iris scanner software then checks to see if this is a returning user, greeting a returning customer by name or creating a new customer record by asking a user for his or her information if the user is new.

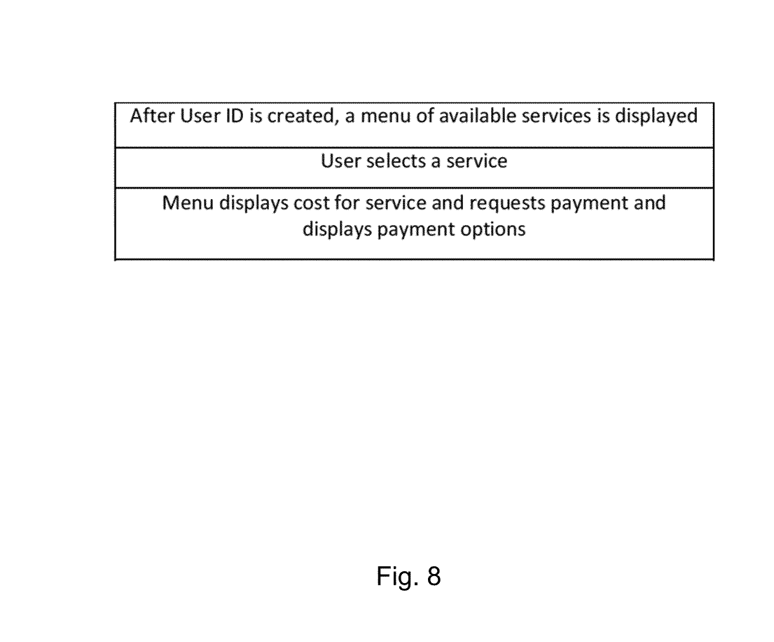

In embodiments and depicted in FIG. 8, after a user ID is created, a menu of available services or items to order may then be displayed. The user may then select a service or item, and the menu displays costs of services or items and requests payment for the selected service or item, displaying payment options.

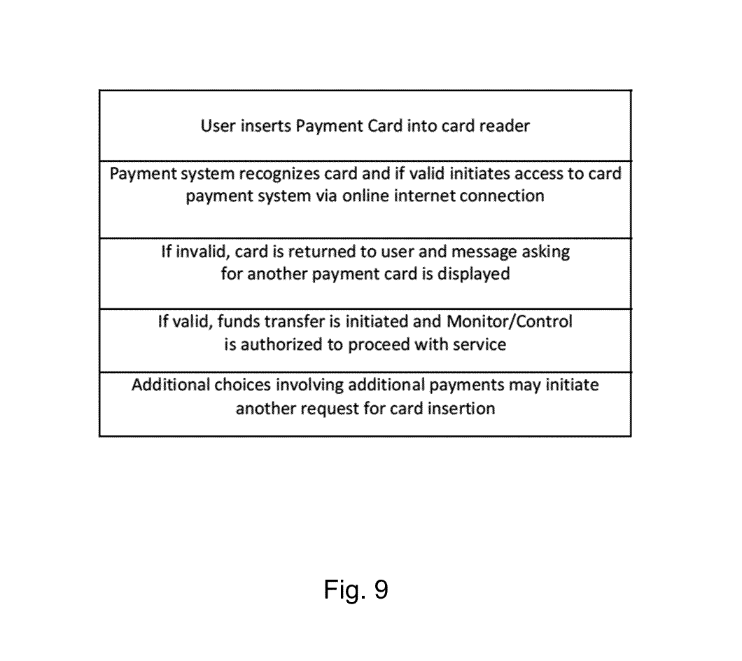

In embodiments and depicted in FIG. 9, a user may insert a payment card into a card reader. The payment system may recognize a card and, if the card is valid, initiate access to a card payment system via a network connection, such as an Internet connection. If invalid, the card is returned to a user and the interface displays a message asking for another form of payment. If the card is valid, the service or item may be provided and the funds transfer may be initiated. Additional choices of services or items involving additional payments may initiate another request for card insertion.

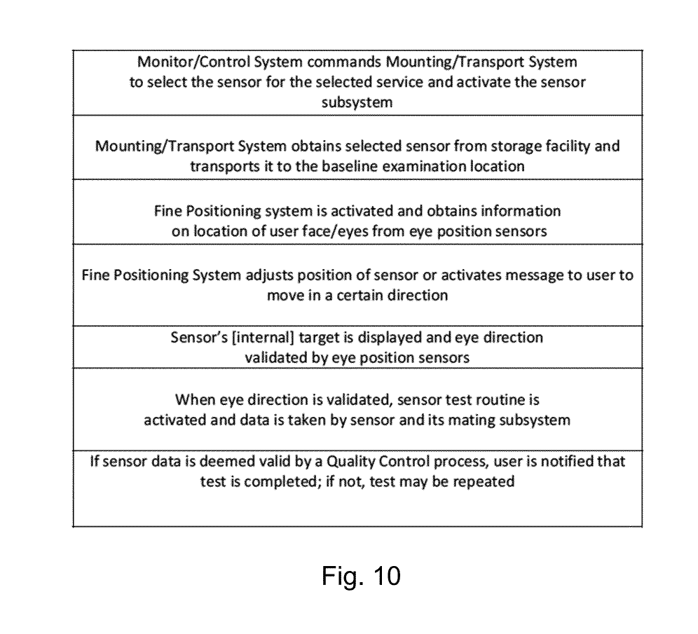

In embodiments and in FIG. 10, the methods and systems disclosed herein may comprise selecting a sensor for a selected service and activating the sensor subsystem. The selected sensor may be obtained from a storage facility and transported to the examination location, where a positioning system is activated to obtain information on a user's face or eyes from position sensors. This may be accomplished by an automated, electro-mechanical system, under the control of a processor, such as a system involving one or more robotic arm components, a system using a carousel, or the like, as described in connection with various embodiments described herein or as known to those of ordinary skill in the art. The sensor may then be adjusted to the user's position or a message may be displayed telling the user to move in a certain direction. A sensor's target may then be displayed and eye direction may be validated by eye position sensors. When eye direction is validated, the sensor test routine is activated and data is taken by sensor and it's mating subsystem. If the sensor data is deemed valid by a quality control process, a user is notified that the test is completed, or the test may be repeated.

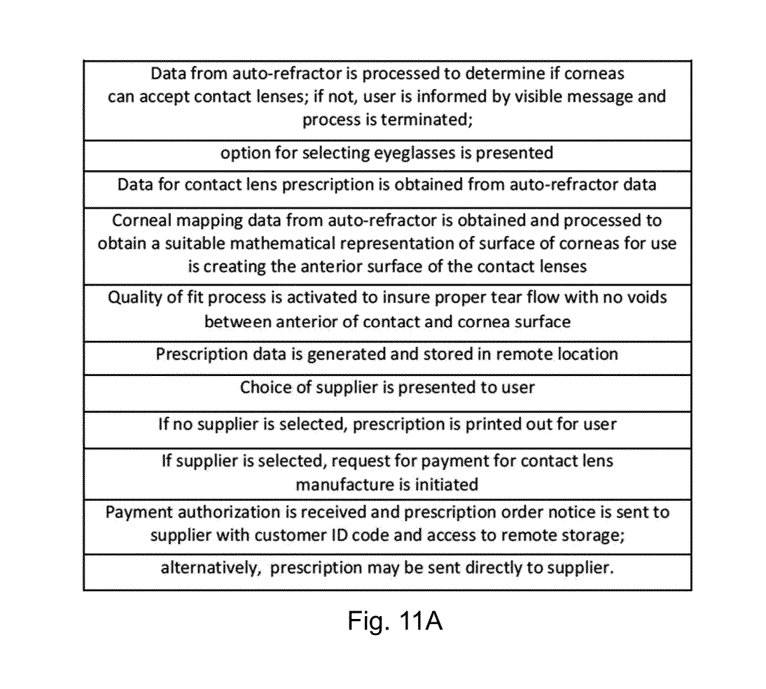

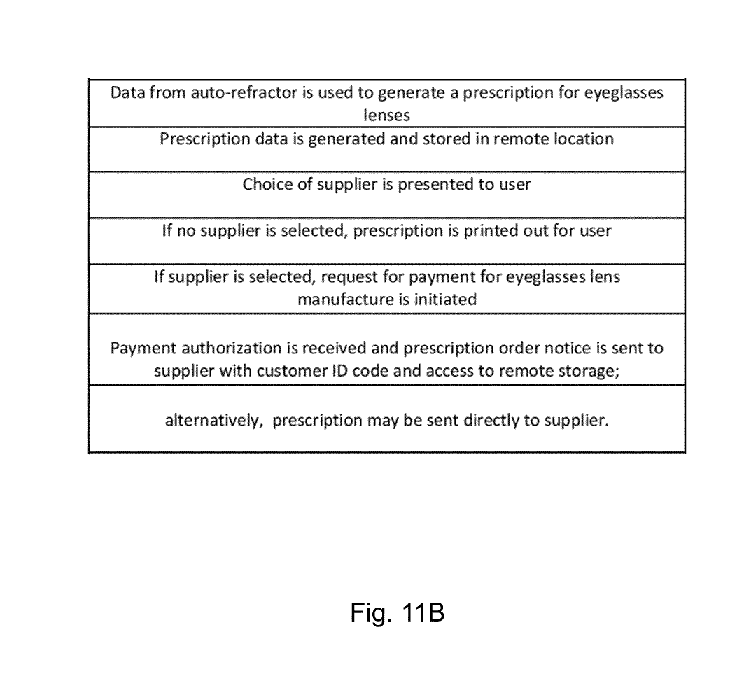

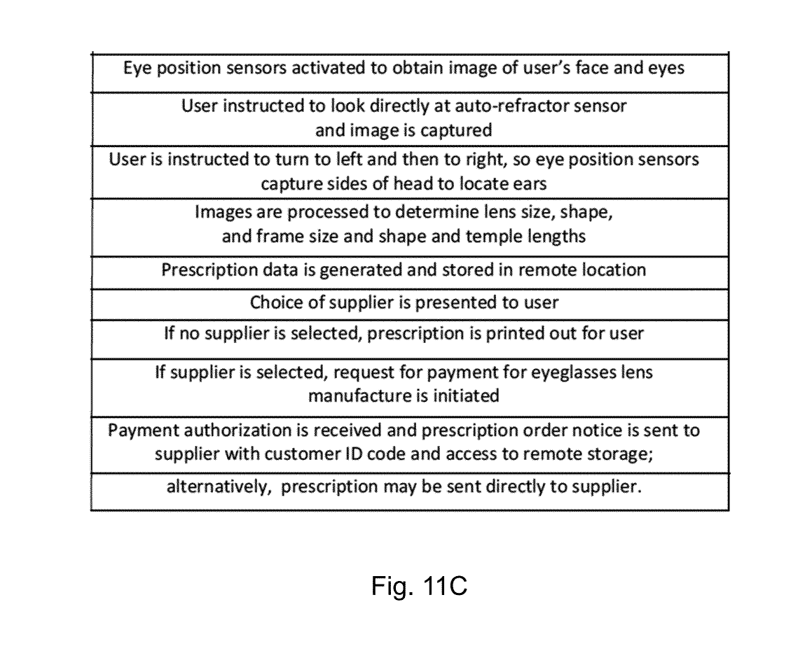

In embodiments and in FIGS. 11A and 11B, the methods and systems disclosed herein may comprise processing data from an auto-refractor to determine if corneas can accept contact lenses; if not, a user is informed by a visible message and the prescription generation for contact lenses is terminated, presenting an option for selecting eyeglasses. Data for contact lens and eye glasses prescriptions is obtained from auto-refractor data along with corneal mapping data and processed to obtain a suitable mathematical representation of surface of corneas for use. The quality of the fit process is activated to insure proper tear flow and the absence of voids between anterior of contact and cornea surface. The prescription data may then be generated and stored in a remote location. A choice of a supplier may then be presented to a user. If the user does not choose a supplier, a prescription is printed out for a user. If a supplier is selected, a request for payment is initiated, payment authorization is received, and the supplier receives prescription order notice with customer ID code and access to remote storage. Alternatively, the prescription may be sent directly to a supplier.

In embodiments and in FIG. 11C, a prescription for an eye glasses frame may be generated by using eye position sensors activated to obtain image of a user's face and eyes. A user may then be instructed to look directly at auto-refractor sensor, and an image is captured. The user may then be instructed to turn in different positions so that eye position sensors capture various facial landmarks, such as location of ears, size of head, etc. Images may then be processed to determine lens size, shape, and frame size and shape and temple lengths. The prescription data may then be generated and stored in a remote location. A choice of a supplier may then be presented to a user. If the user does not choose a supplier, a prescription is printed out for a user. If a supplier is selected, a request for payment is initiated, payment authorization is received, and the supplier receives prescription order notice with customer ID code and access to remote storage. Alternatively, the prescription may be sent directly to a supplier.

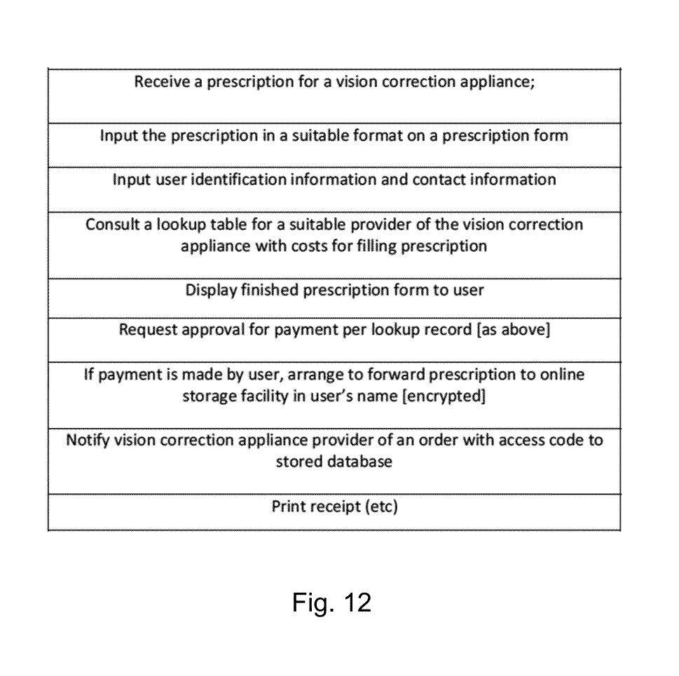

In embodiments and in FIG. 12, the systems and methods disclosed herein may comprise report preparation and delivery. In embodiments a prescription for a vision correction appliance is received and is inputted in a suitable format on a prescription form. User identification and contact information may also be inputted. A suitable provider's lookup table is consulted for the vision correction appliance with costs for filling a prescription. The prescription form may then be displayed to a user, requesting approval for payment per the lookup record. If payment is made by the user, the prescription may be forwarded in an encrypted format to an online storage facility in user's name. A vision correction appliance provider may then be notified of an access code to stored database and a receipt may be printed.

While only a few embodiments of the present invention have been shown and described, it will be obvious to those skilled in the art that many changes and modifications may be made thereunto without departing from the spirit and scope of the present invention as described in the following claims. All patent applications and patents, both foreign and domestic, and all other publications referenced herein are incorporated herein in their entireties to the full extent permitted by law.

While the invention has been described in connection with certain preferred embodiments, other embodiments would be understood by one of ordinary skill in the art and are encompassed herein.

The methods and systems described herein may be deployed in part or in whole through a machine that executes computer software, program codes, and/or instructions on a processor. The present invention may be implemented as a method on the machine, as a system or apparatus as part of or in relation to the machine, or as a computer program product embodied in a computer readable medium executing on one or more of the machines. The processor may be part of a server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic coprocessor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more thread. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer software on a server, client, firewall, gateway, hub, router, or other such computer and/or networking hardware. The software program may be associated with a server that may include a file server, print server, domain server, internet server, intranet server and other variants such as secondary server, host server, distributed server and the like. The server may include one or more of memories, processors, computer readable media, storage media, ports (physical and virtual), communication devices, and interfaces capable of accessing other servers, clients, machines, and devices through a wired or a wireless medium, and the like. The methods, programs or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the invention. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client and other variants such as secondary client, host client, distributed client and the like. The client may include one or more of memories, processors, computer readable media, storage media, ports (physical and virtual), communication devices, and interfaces capable of accessing other clients, servers, machines, and devices through a wired or a wireless medium, and the like. The methods, programs or codes as described herein and elsewhere may be executed by the client. In addition, other devices required for execution of methods as described in this application may be considered as a part of the infrastructure associated with the client.

The client may provide an interface to other devices including, without limitation, servers, other clients, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the invention. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium such as flash memory, buffer, stack, RAM, ROM and the like. The processes, methods, program codes, instructions described herein and elsewhere may be executed by one or more of the network infrastructural elements.

The methods, program codes, and instructions described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like. The cell network may be a GSM, GPRS, 3G, EVDO, mesh, or other networks types.

The methods, programs codes, and instructions described herein and elsewhere may be implemented on or through mobile devices. The mobile devices may include navigation devices, cell phones, mobile phones, mobile personal digital assistants, laptops, palmtops, netbooks, pagers, electronic books readers, music players and the like. These devices may include, apart from other components, a storage medium such as a flash memory, buffer, RAM, ROM and one or more computing devices. The computing devices associated with mobile devices may be enabled to execute program codes, methods, and instructions stored thereon. Alternatively, the mobile devices may be configured to execute instructions in collaboration with other devices. The mobile devices may communicate with base stations interfaced with servers and configured to execute program codes. The mobile devices may communicate on a peer to peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on machine readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g. USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

The methods and systems described herein may transform physical and/or or intangible items from one state to another. The methods and systems described herein may also transform data representing physical and/or intangible items from one state to another.

The elements described and depicted herein, including in flow charts and block diagrams throughout the figures, imply logical boundaries between the elements. However, according to software or hardware engineering practices, the depicted elements and the functions thereof may be implemented on machines through computer executable media having a processor capable of executing program instructions stored thereon as a monolithic software structure, as standalone software modules, or as modules that employ external routines, code, services, and so forth, or any combination of these, and all such implementations may be within the scope of the present disclosure. Examples of such machines may include, but may not be limited to, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipment, servers, routers and the like. Furthermore, the elements depicted in the flow chart and block diagrams or any other logical component may be implemented on a machine capable of executing program instructions. Thus, while the foregoing drawings and descriptions set forth functional aspects of the disclosed systems, no particular arrangement of software for implementing these functional aspects should be inferred from these descriptions unless explicitly stated or otherwise clear from the context. Similarly, it will be appreciated that the various steps identified and described above may be varied, and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

The methods and/or processes described above, and steps thereof, may be realized in hardware, software or any combination of hardware and software suitable for a particular application. The hardware may include a general purpose computer and/or dedicated computing device or specific computing device or particular aspect or component of a specific computing device. The processes may be realized in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine-readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

While the invention has been disclosed in connection with the preferred embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

All documents referenced herein are hereby incorporated by reference.

We claim:

1. A system, comprising:
a self-contained, standing housing, configured to include an interface for a user;
at least one vision assessment facility integrated with the housing, the vision assessment facility capable of determining a prescription for at least one of contact lenses and eyeglasses for the user;
at least one health assessment facility related to a non-vision aspect of the health of a user integrated with the housing.

2. A system of claim 1, wherein the vision assessment facility automatically aligns with the eyes of the user, without requiring a mechanical element for positioning the head of the user relative to the vision assessment facility.

3. A system of claim 1, further comprising a vision recommendation for analyzing a need identified by the vision assessment facility and recommending at least one of an item and an action to address the need.

4. A system of claim 1, wherein the housing is configured as a kiosk adapted to be located in a retail location.

5. A system of claim 1, wherein the system includes a network communication facility.

6. A system of claim 1, further comprising a health recommendation module for analyzing a need identified by the health assessment facility and recommending at least one of an item and an action to address the need.

7. A system of claim 1, wherein the system includes a plurality of health assessment facilities, wherein at least two of the facilities are disposed on a rotating carousel to allow serial presentation of the facilities to the user.

8. A system of claim 1, wherein the system includes a plurality of health assessment facilities, wherein at least two of the facilities are disposed to allow presentation of the facilities to the user without requiring a rotating carousel.

9. A system of claim 1, wherein the health assessment facility is selected from the group consisting of a contact lens auto refractor, a corneal mapper, a corneal spline generator, a retinal macular condition sensor, a retinal circulatory physiology sensor, a 3D surface scanner, a glaucoma sensor, a blood pressure monitor, a pulse rate monitor, a diabetes sensor, and an iris scan sensor.

10. A system of claim 1, further comprising a payment system by which a user may pay for at least one of an assessment, a recommended item, and a recommended action.

11. A network-connected, retail kiosk, comprising:
a plurality of health assessment facilities, each adapted to assess a health condition of a user;
a vision assessment facility capable of determining a contact lens prescription and an eyeglass prescription of a user, the vision assessment facility adapted to align with the eyes of the user and assess vision while the head of the user remains in a natural, unconstrained position;
a recommendation module for recommending at least one of an item and an action based on at least one of a health assessment and a vision assessment;
an electronic commerce module for ordering a recommended item; and
a scheduling module for scheduling a recommended action.

12. A system of claim 11, wherein the health assessment facility is selected from the group consisting of a contact lens auto refractor, a corneal mapper, a corneal spline generator, a retinal macular condition sensor, a retinal circulatory physiology sensor, a 3D surface scanner, a glaucoma sensor, a blood pressure monitor, a pulse rate monitor, a diabetes sensor, and an iris scan sensor.

13. A computer readable medium containing program instructions wherein execution of the program instructions by one or more processors of a computer system causes the one or more processors to carry out the steps of:
conducting a vision assessment of a user via at least one vision assessment facility capable of determining a prescription for at least one of contact lenses and eyeglasses for the user;
conducting a health assessment of a user via at least one health assessment facility related to a non-vision aspect of the health of a user;
storing data obtained from the vision assessment and health assessment on a memory device;
retrieving, in response to a user request via a user interface, requested vision assessment and health assessment data; and
presenting the retrieved vision assessment and health assessment data to the user via the user interface.

14. The computer readable medium of claim 13, further comprising automatically aligning the vision assessment facility with the eyes of a user, without requiring a mechanical element for positioning the head of the user relative to the vision assessment facility.

15. The computer readable medium of claim 14, further comprising conducting a vision recommendation for a need identified by the vision assessment and recommending at least one of an item and an action to address the need.

16. The computer readable medium of claim 15, further comprising processing a payment by a user to order a recommended item.

17. The computer readable medium of claim 15, further comprising scheduling an appointment with an eye specialist.

18. The computer readable medium of claim 13 further comprising conducting a health recommendation for a need identified by the health assessment and recommending at least one of an item and an action to address the need.

19. The computer readable medium of claim 18, further comprising processing a payment by a user to order a recommended item.

20. The computer readable medium of claim 18, further comprising scheduling an appointment to address a recommended item.
